(19) United States
(12) Patent Application Publication
(10) Pub. No.: US 2024/0358298 A1
(43) Pub. Date: Oct. 31, 2024

(54) DEVICES AND METHODS FOR SAMPLE COLLECTION

(71) Applicant: ZYMO RESEARCH CORPORATION, Irvine, CA (US)

(72) Inventors: Ian FORSYTHE, Irvine, CA (US); Marc E. VAN EDEN, Irvine, CA (US); Stanislav FORMAN, Irvine, CA (US); Xi-Yu JIA, Irvine, CA (US)

(73) Assignee: ZYMO RESEARCH CORPORATION, Irvine, CA (US)

(21) Appl. No.: 18/681,948

(22) PCT Filed: Aug. 12, 2022
(86) PCT No.: PCT/US22/74898

§ 371 Date: Feb. 7, 2024

Related U.S. Application Data
(60) Provisional application No. 63/232,899, filed on Aug. 13, 2021.

(51) Int. Cl. A61B 5/15; A61B 10/00; A61B 10/02

(52) U.S. Cl. CPC A61B 5/150343; A61B 5/150274; A61B 5/150351; A61B 10/0038; A61B 10/0051; A61B 10/02

(57) ABSTRACT

Exemplary embodiments include a sample collection device having a tube with an inner seal producing a sequestered lower compartment. In certain embodiments, the sequestered compartment may have a stabilizing reagent. The device may also have a cap suited towards the sample type being stored in the tube. In some cases, the cap may pierce the inner seal to allow a liquid sample to mix with the stabilizing reagent. In the case of liquid samples, a funnel may be used to aid in the deposition of the sample. In other cases, a swab or similar collection tool can be inserted into the tube, piercing the inner seal, and the cap will retain the swab in the cap. The device can be used for the collection of saliva, swab, or other samples at home without clinical supervision. The samples can be transported and stored for further analysis of the sample for DNA, RNA, or other biological components.

### CROSS-REFERENCE TO RELATED APPLICATION

This application claims priority to U.S. Provisional Patent Application Ser. No. 63/232,899, filed Aug. 13, 2021, the entire contents of which are incorporated herein by reference.

### BACKGROUND OF THE INVENTION

Embodiments of the present invention relate generally to the field of sample collection. Particular embodiments relate to devices and methods that include aspects to improve sample collection and manufacturing of devices configured for sample collection.

Biological samples can provide invaluable information for scientists, physicians, epidemiologists, and geneticists, among others. For example, collection of these samples can provide access to information for a physician to identify the cause of an infection or for a scientist to conduct genetic based studies. Yet, collecting these samples can be inconvenient and burdensome for the average individual, as traveling to a collection center with the appropriate laboratory conditions and equipment or directly accessing patients can be unfeasible.

To address this, present sample collection devices are sent to donors for samples to be collected in their home. Collection devices sent to donors for home collection are then shipped back to the laboratory or medical facility for further analysis. Such devices often include stabilization reagents to maintain the viability of the sample as it is shipped back to a laboratory or stored prior to analysis.

However, the kits or devices provided for sample collection are often implemented by individuals lacking training or experience with utilizing the device and depositing samples into the collection device. This can cause a multitude of issues, including, but not limited to, the preservation or accuracy of the biological sample. In many cases, if a user mishandles a collection device, they will need to be sent a new device to collect a viable sample. In other cases, customers unfamiliar with sample collection may incorrectly ingest, or otherwise be exposed to, stabilization reagents contained in sample collection devices. With the lack of training and experience, it is important to have a sample collection device that is feasible for use and access for the average individual.

Therefore, there is an opportunity to address these issues and overcome the present disadvantages.

### SUMMARY OF THE INVENTION

Embodiments disclosed herein include a sample collection device and associated methods. In certain embodiments the sample collection device has a sample collection tube with a lower compartment sealed by a foil seal. In particular embodiments, the lower compartment of the tube contains a stabilizing reagent for stabilizing the samples during transport or storage. Different caps can be available for use with the tube, depending on the sample type to be collected with the tube. Embodiments of the invention are useful for the collection of saliva samples, swab samples, and other sample types. The reagent included in the lower compartment of the tube can stabilize the contents of the sample (DNA/RNA in some embodiments). The invention is useful for the at-home collection of biological samples in some embodiments.

The device can be used without clinical supervision, due to an easy-to-use design. The inner seal of the tube prevents the user from spilling the reagent stored in the lower compartment of the tube until the seal is pierced. In some cases, the seal is pierced by a cap designed to pierce the inner seal. In other cases, the seal is pierced by a swab (or similar collection device), in specific cases the cap captures the swab. In these embodiments, the seal is broken to allow the sample to mix with or submerge into the stabilizing reagent in the lower compartment.

Exemplary embodiments include a device for collecting a sample where the device comprises a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and a plurality of ribs coupling the first outer wall portion and the second outer wall portion to the inner wall portion, where: the plurality of ribs comprises a plurality of central ribs each having planar sides and a curved end; the plurality of ribs comprises a first outer rib having a flat side and a curved side; the plurality of ribs comprises a second outer rib having a flat side and a curved side; the plurality of ribs comprises a first perpendicular rib coupled to the flat side of the first outer rib and the inner wall portion; and the plurality of ribs comprises a second perpendicular rib coupled to the flat side of the second outer rib and the inner wall portion; the inner wall portion comprises a tapered closed end proximal to the second end of the cylindrical tube; and the inner wall portion comprises an open end proximal to the first end of the cylindrical tube; and a pierceable seal coupled to the open end of the inner wall portion.

Particular embodiments further comprise a cap configured to couple to the first end of the cylindrical tube, and certain embodiments comprise a sample collector coupled to the cap. In some embodiments, the sample collector as a flocked swab head. In other embodiments, the sample collector as a grooved collection head, and in specific embodiments the sample collector as a collection head with an angular step design. In certain embodiments the inner wall portion, pierceable seal and tapered closed end form a sealed compartment, and in particular embodiments the sealed compartment comprises a reagent.

In specific embodiments, each central rib is tapered such that a thickness of each central rib at the curved end is less than a thickness of each central rib proximal to the inner wall portion, and in some embodiments, each planar side of each central rib is tapered 1 degree. In particular embodiments, the cap is configured to couple to the first end of the cylindrical tube via a threaded coupling. In certain embodiments the cap comprises a piercing element configured to pierce the pierceable seal when the cap is coupled to the first end of the cylindrical tube. In particular embodiments, the piercing element has a circular or semi-circular cross-section, and in some embodiments the circular or semi-circular cross-section is configured to form a circular cutout of pierceable seal.

In some embodiments the circular or semi-circular cross-section is hollow. In specific embodiments the piercing element has an end with an angled surface. In particular embodiments the cap comprises a ledge configured to seal to the cylindrical tube. In some embodiments the piercing element has a length of approximately 25 mm to approximately 26 mm. In specific embodiments the cap comprises threads configured to engage the cylindrical tube. In certain embodiments the ledge is configured seal to the cylindrical tube prior to the piercing element piercing the pierceable seal when the cap is threadably engaged with the cylindrical tube

In some embodiments an arc of about 20 to about 45 degrees is attached to the sealing surface. In specific embodiments, the piercing element comprises an angled surface configured to push aside the circular cutout.

In particular embodiments the plurality of ribs comprise lateral ribs. In some embodiments the plurality of ribs form a checkerboard pattern. In specific embodiments the ribs are eliminated in favor of a gap between the inner wall portion and outer wall portion to form the sealing surface. In certain embodiments the closed end is tapered. In particular embodiments the closed end comprises a flat bottom. In some embodiments the closed end comprises an identifying code. Specific embodiments further comprise a tube capsule inserted into the cylindrical tube. In some embodiments the tube capsule is filled with a reagent. In certain embodiments the tube capsule comprises an inner wall portion and an outer wall portion; and the outer wall portion of the tube capsule is inserted into the inner wall portion of the cylindrical tube. In particular embodiments the tube capsule comprises an open end and closed end. In some embodiments the open end of the tube capsule is sealed In specific embodiments the open end of the tube capsule is sealed via ultrasonic welding, laser welding, adhesives, press fit, or threaded fit.

Exemplary embodiments also include a method of collecting a sample, where the method comprises: obtaining a device comprising: a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and a plurality of ribs coupling the first outer wall portion and the second outer wall portion to the inner wall portion, where: the plurality of ribs comprises a plurality of central ribs each having planar sides and a curved end; the plurality of ribs comprises a first outer rib having a flat side and a curved side; the plurality of ribs comprises a second outer rib having a flat side and a curved side; the plurality of ribs comprises a first perpendicular rib coupled to the flat side of the first outer rib and the inner wall portion; and the plurality of ribs comprises a second perpendicular rib coupled to the flat side of the second outer rib and the inner wall portion; the inner wall portion comprises a tapered closed end proximal to the second end of the cylindrical tube; and the inner wall portion comprises an open end proximal to the first end of the cylindrical tube; a pierceable seal coupled to the open end of the inner wall portion; and a cap configured to couple to the first end of the cylindrical tube; and depositing a sample into the first end of the cylindrical tube; and coupling the cap to the first end of the cylindrical tube.

In certain embodiments of the method, the device further comprises a funnel; and depositing the sample into the first end of the cylindrical tube comprises: coupling the funnel to the first end of the cylindrical tube; and depositing the sample into the funnel. In particular embodiments of the method, the cap comprises a piercing element configured to pierce the pierceable seal when the cap is coupled to the first end of the cylindrical tube; and coupling the cap to the first end of the cylindrical tube pierces the pierceable seal. In some embodiments, the inner wall portion contains a reagent between the pierceable seal and the tapered closed end; and coupling the cap to the first end of the cylindrical tube pierces the pierceable seal and mixes the sample with the reagent. Specific embodiments further comprise inverting the cylindrical tube to further mix the sample with the reagent, and in particular embodiments, the reagent stabilizes the sample.

Exemplary embodiments include a method of manufacturing a cylindrical tube, the method comprising: injecting a liquid polymer at a first temperature into a mold partially comprised of a split mold, wherein the split mold comprises a first half and a second half coupled at a split line; reducing the first temperature of the liquid polymer injected into the split mold to a second temperature, wherein the liquid polymer injected into the split mold forms a solid polymer component at the second temperature; separating the first half of the split mold and the second half of the split mold at the parting line; removing the solid polymer component from the split mold, wherein the solid polymer component is a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and a plurality of ribs coupling the first outer wall portion and the second outer wall portion to the inner wall portion, where: the plurality of ribs comprises a plurality of central ribs each having planar sides and a curved end; the plurality of ribs comprises a first outer rib having a flat side and a curved side; the plurality of ribs comprises a second outer rib having a flat side and a curved side; the plurality of ribs comprises a first perpendicular rib coupled to the flat side of the first outer rib and the inner wall portion; and the plurality of ribs comprises a second perpendicular rib coupled to the flat side of the second outer rib and the inner wall portion; the inner wall portion comprises a tapered closed end proximal to the second end of the cylindrical tube; and the inner wall portion comprises an open end proximal to the first end of the cylindrical tube.

Certain embodiments include a device for collecting a blood sample, the device comprising: a cylindrical tube comprising: a first open end; a second closed end; an outer wall portion proximal to the first open end of the cylindrical tube; an inner wall portion proximal to the second closed end and coupled to the outer wall portion, wherein the inner wall portion comprises an open end proximal to the first end of the cylindrical tube; and a plurality of ribs extending from the closed end of the inner wall portion to the outer wall portion; and a pierceable seal coupled to the open end of the inner wall portion. Particular embodiments further comprise a cap comprising a hollow circular or semi-circular piercing element configured to pierce the pierceable seal when the cap is coupled to the first end of the cylindrical tube, and wherein the piercing element has a length of approximately 15 mm to approximately 16 mm. In some embodiments the piercing element has an end with an angled surface. In specific embodiments the piercing element is configured to form a cutout in the pierceable seal when the cap is coupled to the first end of the cylindrical tube; and the angled surface is configured to push aside the cutout in the pierceable seal. In certain embodiments the hollow circular or semi-circular piercing element is configured to form an approximately circular cutout of the pierceable seal, wherein an arc of about 20 to about 45 degrees is attached to the sealing surface.

In particular embodiments an arc of about 20 to about 45 degrees is attached to the sealing surface. In some embodiments the piercing element comprises an angled surface configured to push aside the circular cutout. In specific embodiments the plurality of ribs comprise lateral ribs. In certain embodiments the plurality of ribs form a checkerboard pattern. In particular embodiments the ribs are eliminated in favor of a gap between the inner wall portion and outer wall portion to form the sealing surface. In some embodiments the closed end is tapered. In specific embodiments the closed end comprises a flat bottom. In certain embodiments the closed end comprises an identifying code. Particular embodiments further comprise a tube capsule inserted into the cylindrical tube. In some embodiments the tube capsule is filled with a reagent. In specific embodiments the tube capsule comprises an inner wall portion and an outer wall portion; and the outer wall portion of the tube capsule is inserted into the inner wall portion of the cylindrical tube. In particular embodiments the tube capsule comprises an open end and closed end. In some embodiments the open end of the tube capsule is sealed. In certain embodiments the open end of the tube capsule is sealed via ultrasonic welding, laser welding, adhesives, press fit, or threaded fit.

Certain embodiments include device for collecting a fecal sample, the device comprising: a cylindrical tube comprising: a first open end; a second closed end; a first outer wall portion spanning the first and second end of the cylindrical tube; a second inner wall comprising a cylindrical tube comprising: a first open end; a second closed end; a wall spanning the first and second end; and the cylindrical tube further comprising an inner wall portion inserted into the cylindrical tube comprising the outer wall portion, wherein the inner wall portion is coupled to the outer wall portion of the cylindrical tube, a pierceable seal coupled to the open end of the cylindrical tube comprising the inner wall portion. Particular embodiments further comprise a cap configured to couple to the first end of the cylindrical tube, wherein the cap further comprises a sample collector coupled to the cap. In some embodiments the sample collector is a spoon or scoop.

Certain embodiments include device for collecting a fecal sample, the device comprising: a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and a plurality of ribs coupling the first outer wall portion and the second outer wall portion to the inner wall portion, wherein: the plurality of ribs comprises a plurality of central ribs each having planar sides and a curved end; the plurality of ribs comprises a first outer rib having a flat side and a curved side; the plurality of ribs comprises a second outer rib having a flat side and a curved side; the plurality of ribs comprises a first perpendicular rib coupled to the flat side of the first outer rib and the inner wall portion; the plurality of ribs comprises a second perpendicular rib coupled to the flat side of the second outer rib and the inner wall portion; the inner wall portion comprises a closed end proximal to the second end of the cylindrical tube; the inner wall portion comprises an open end proximal to the first end of the cylindrical tube; a pierceable seal coupled to the open end of the inner wall portion; a wall spanning the first and the second end; and the cylindrical tube further comprising an inner wall portion inserted into the cylindrical tube comprising the outer wall portion, wherein the inner wall portion is coupled to the outer wall portion of the cylindrical tube, a pierceable seal coupled to the open end of the cylindrical tube comprising the inner wall portion. Particular embodiments further comprise a cap configured to couple to the first end of the cylindrical tube, wherein the cap further comprises a sample collector coupled to the cap. In some embodiments the sample collector is a spoon or scoop.

Specific embodiments include a device for collecting a sample, the device comprising: a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and the inner wall portion comprises a closed end proximal to the second end of the cylindrical tube; and the inner wall portion comprises an open end proximal to the first end of the cylindrical tube; and a pierceable seal coupled to the open end of the inner wall portion and of a gap between the inner wall portion and outer wall portion to form the sealing surface.

Certain embodiments include a device for collecting a sample, the device comprising: a cylindrical tube comprising: a first end; a second end; a first outer wall portion proximal to the first end of the cylindrical tube; a second outer wall portion proximal to the second end of the cylindrical tube; an inner wall portion extending between the first outer wall portion and the second outer wall portion; and the inner wall portion comprises a closed end proximal to the second end of the cylindrical tube; and the inner wall portion comprises an open end proximal to the first end of the cylindrical tube; and a pierceable seal coupled to the open end of the inner wall portion a gap between the inner wall portion and outer wall portion to form the sealing surface; and a tube capsule inserted into the cylindrical tube. In particular embodiments the tube capsule is filled with a reagent. In some embodiments the tube capsule comprises an inner wall portion and an outer wall portion; and the outer wall portion of the tube capsule is inserted into the inner wall portion of the cylindrical tube. In specific embodiments the tube capsule comprises an open end and closed end. In certain embodiments the open end of the tube capsule is sealed. In particular embodiments the open end of the tube capsule is sealed via ultrasonic welding, laser welding, adhesives, press fit, or threaded fit.

As used in this specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about”, “approximately” or related terms are used to indicate that a value includes the inherent variation of error for the device, the method being employed to determine the value, or the variation that exists among the study subjects.

As used herein, the term “binding” or “binding buffer” includes chemistries to bind nucleic acids to a mobile surface such as magnetizable bead or resins. Example chemistries include chaotropic driven adsorption, cationic detergent driven nucleic acid capture, PEG/Cellulose combinations, PEG/Carboxylated beads commonly referred to as SPRI technology, chitosan driven binding and other precipitation and adsorption based methods that allow nucleic acids to bind to a mobile surface.

As used herein, the term “sample preparation” (and variants thereof) includes sample purification, isolation, immunoprecipitation, and other similar techniques.

Any embodiment of any of the present methods, kits, and compositions may consist of or consist essentially of—rather than comprise/include/contain/have—the described features and/or steps. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1-3 illustrate front and section views of a device according to an exemplary embodiment of the present disclosure where in an inner and outer wall with peripheral ribs forms a sealing surface for the application of a circular seal.

FIG. 4 illustrates perspective and front views of the embodiment of FIGS. 1-3 with additional components including a funnel.

FIG. 5 illustrates perspective and front views of the embodiment of FIGS. 1-3 with additional components including a swab.

FIG. 6 illustrates front and section views of a device according to an exemplary embodiment of the present disclosure comprising a false bottom.

FIG. 7 illustrates front and section views of alternate embodiments of a device according to an exemplary embodiment of the present disclosure comprising lateral ribs.

FIG. 8 illustrates perspective and front views of a seal and material used to form the seal.

FIG. 9 illustrates perspective views of alternative methods for manufacturing an internal sequestration feature of a device according to an exemplary embodiment of the present disclosure.

FIG. 10 illustrates front and perspective views of alternate embodiments of a device according to an exemplary embodiment of the present disclosure with additional features to accommodate certain methods of automation including the addition of 2D codes for accessioning.

FIG. 11 illustrates perspective, top and front views of a device according to an exemplary embodiment of the present disclosure interfacing with a rack for storage, transport, reading of 2D codes, and handling.

FIG. 12 illustrates front and section views of an embodiment of a cap with a piercing element according to the present disclosure.

FIG. 13 illustrates a section view of an embodiment of a tube in which a piercing cap wherein a piercing element has pierced the seal.

FIG. 14 illustrates front, side and perspective views of alternative embodiments of a piercing cap design that incorporate additional functionality.

FIG. 15 illustrates front, section and perspective views of additional embodiments of a piercing cap designed for the precise sample collection of specific sample types.

FIG. 16 illustrates a front view of a funnel that can be used to collect a sample.

FIG. 17 illustrates front and section views of a cap design based on prior art.

FIG. 18 illustrates a front view of a swab with a swab head according to an exemplary embodiment of the present disclosure.

FIG. 19 illustrates front views of a plurality of adhesive swabs for skin or surfaces according to exemplary embodiments of the present disclosure.

FIG. 20 illustrates exploded and perspective views of an adhesive roller swabs according to exemplary embodiments of the present disclosure for the collection of a sample.

FIG. 21 illustrates front views of swab-based molded sample collectors for collecting a defined volume of sample.

FIG. 22 illustrates front and section views of an embodiment of a cap and exemplary molded swab produced as a single part according to the present disclosure.

FIG. 23 illustrates front, section and perspective views of an embodiment of the present disclosure that has been miniaturized to collect small amounts of blood.

FIG. 24 illustrates embodiments of the present disclosure with different colored media and the results of piercing cap mixing test showing the superior function of the illustrated embodiments

FIG. 25 illustrates an embodiment of the present disclosure with a modified version of a piercing cap with an extended piercing element.

FIG. 26 illustrates a graph of percent seal failures for different seal materials.

### DESCRIPTION OF ILLUSTRATIVE EMBODIMENTS

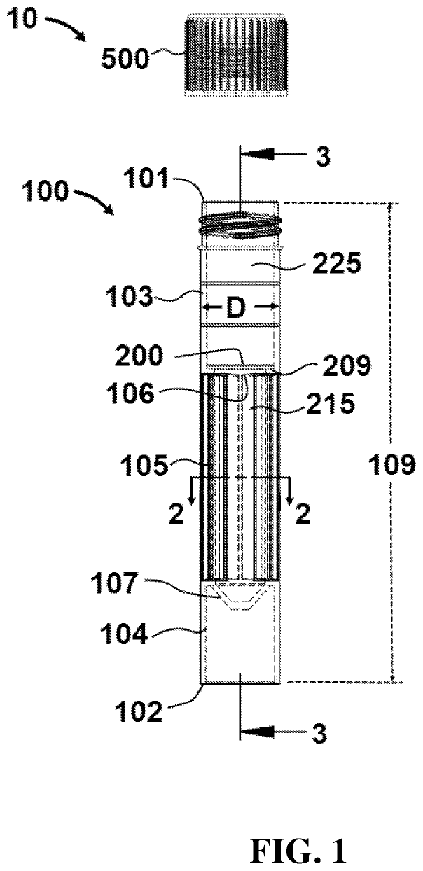

Referring initially to FIGS. 1-3 side and section views of a device 10 for collecting a sample are shown. In this embodiment, device 10 comprises a cylindrical tube 100 (also referred to herein as the “SafeCollect®™ tube”) and a cap 500. It is understood that some embodiments of device 10 may comprise cylindrical tube 100 without cap 500. Cylindrical tube 100 further comprises a first end 101, a second end 102, a first outer wall portion 103 proximal to first end 101, and a second outer wall portion 104 proximal to second end 102. Cylindrical tube 100 also comprises an inner wall portion 105 extending between first outer wall portion 103 and second outer wall portion 104.

In addition, the illustrated embodiment comprises a plurality of ribs coupling first outer wall portion 103 and second outer wall portion 104 to inner wall portion 105. The particular embodiment shown comprises a plurality of central ribs 110, a first outer rib 130 a second outer rib 140. In this embodiment central ribs 110 each have planar sides 111 and a curved end 112, while first outer rib 130 has a flat side 131 and a curved side 132 and second outer rib 140 has a flat side 141 and a curved side 142. The illustrated embodiment also comprises a first perpendicular rib 135 coupled to flat side 131 of first outer rib 130 and inner wall portion 105, and comprises a second perpendicular rib 145 coupled to flat side 141 of second outer rib 140 and inner wall portion 105.

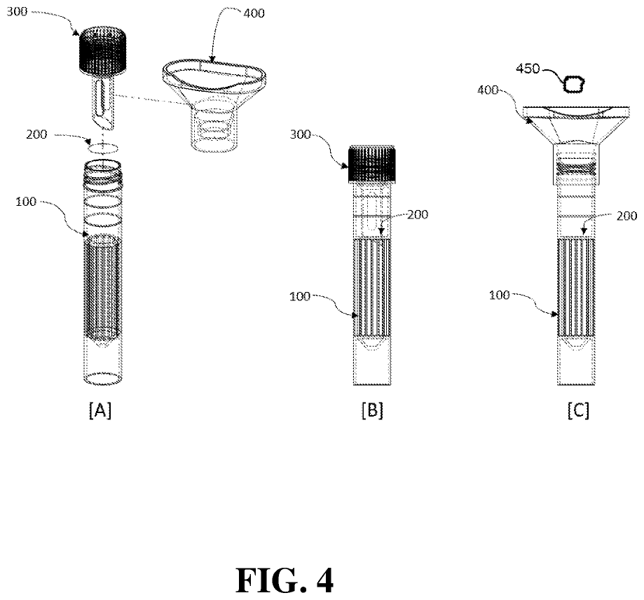

In the embodiment shown, inner wall portion 105 comprises a tapered closed end 107 proximal to second end 102 of cylindrical tube 100. Inner wall portion 105 also comprises an open end 106 proximal to first end 101 of cylindrical tube. The illustrated embodiment also comprises a pierceable seal 200 coupled to open end 106 of inner wall portion 105 on a sealing surface 209. In the embodiment shown, cap 500 is configured to couple to first end 101 of cylindrical tube 100 via a threaded coupling 115, but other embodiments may incorporate alternate coupling mechanisms, including for example, a snap or press fit. Cap 500 can be coupled to end 101 of tube 100 to seal sample 450 from the outside environment. Tube 100 containing sample 450 (FIG. 4) can then be stored or transported if desired.

In exemplary embodiments, the open end 106 of inner wall portion 105 provides a sealing surface 209 for pierceable seal 200. In the illustrated embodiment, the application of a pierceable seal 200 to the sealing surface 209 generates an upper compartment 160, and lower sealed compartment 170, within the cylindrical tube 100. In exemplary embodiments, the upper compartment 160 of the tube is shown with access to the opening. In certain embodiments, upper compartment 160 connects directly to an opening in first end 101 and holds liquid samples when deposited into the tube. In the illustrated embodiment, the upper compartment 160 can hold over 2 mL of liquid sample. In other embodiments, the upper compartment is of sufficient volume to hold 1 mL, 2 mL, 3 mL, 4 mL, 5 mL, or more of liquid sample.

In the illustrated embodiment, application of seal 200 to sealing surface 209 creates a sealed compartment 170 within inner wall portion 105 between pierceable seal 200 and tapered closed end 107. In certain embodiments, the sealed compartment 170 of the device holds liquid reagent to stabilize the sample 450 collected with the device. In certain embodiments, sealed compartment 170 may comprise one or more reagents, including for example, stabilizing reagents (i.e., Specimen or Viral Transport Media either commercially available or custom made for applications). In such embodiments, the liquid is sealed within the lower portion of the tube by the seal 200. In the illustrated embodiment, the volume of the lower compartment is large enough to hold over 2 mL of stabilizing reagent. In specific embodiments, 500 ul, 1 ml, 1.5 ml or other volumes may be stored in sealed compartment 170 of the tube 100. In other embodiments, alternate dimensions of the tube 100 and sealed compartment 170 may have sufficient volume to hold 1 mL, 2 mL, 3 mL, 4 mL, 5 mL, or more of a liquid reagent.

In one embodiment, the volume of the upper compartment 160 is comparable to the volume of the sealed compartment 170. In other embodiments, the upper compartment and lower sealed compartment of the tube may not have similar volume capacities.

Certain embodiments include an opening in first end 101 of the tube for the insertion of the cap, swab, sample collector, or funnel. In certain embodiments tube comprises threads 115 for screwing on a threaded cap. A variety of caps or sample collectors are designed for the specific sample type.

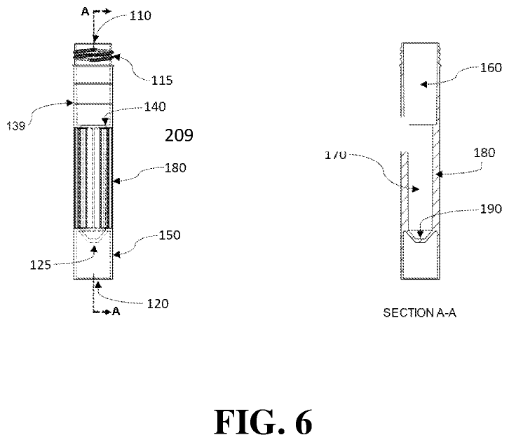

FIG. 6 illustrates an embodiment of the SafeCollect® Tube 100. The length 109 and the diameter D of the illustrated embodiment are informed by preferred tube dimensions within the industry, other embodiments may use different dimensions. The sample tube includes an opening at the top 110 and is threaded 115 for screwing on the cap. In alternate embodiments of the tube 100, internal threading on the interior surface of the tube may be preferred as opposed to the external threading 115 presented. The tube 100 also has an opening at the bottom 120, forming a false bottom or skirted tube design in which the exterior profile of the tube 150 extends beyond the base of the tube cavity 125. In other embodiments, the tube 100 may not use a false bottom. In one embodiment the base of the tube cavity 125 is conical but may be flat in other embodiments to allow for laser etching of bar codes into this region. Other specific embodiments may have a rounded base 125, as opposed to a conical or flat base. The exterior of the tube also includes volumetric markings 139 to indicate when 1 ml or 2 ml of liquid have been added to the tube. Other embodiments may not have volumetric markings.

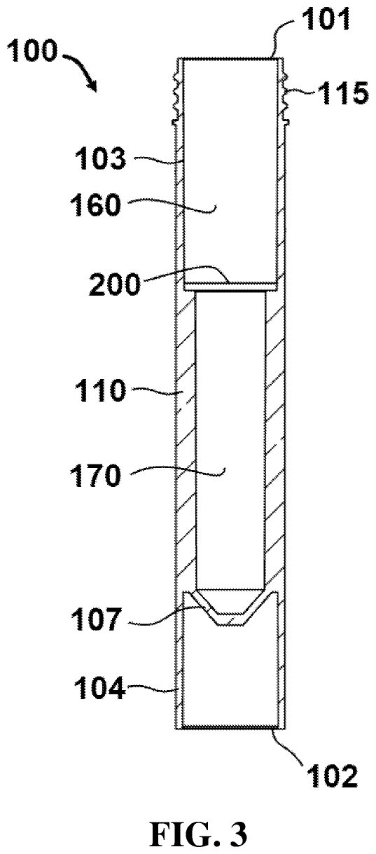

In certain embodiments (e.g. as shown in FIG. 6), the bottom of the tube 100 has a false bottom 120. False bottom tubes are used in automated sample handling, allowing the interior cavity of the tube to be shorter than the exterior of the tube. In some embodiments, the false bottom includes a conical bottom 107 as shown in the section view of FIG. 3. In other embodiments, the bottom 125 of the tube 100 may be altered to allow for laser etching. Certain embodiments may include molded volume markings 139 that highlight the 1 ml and 2 ml volumes in the upper compartment of the tube where liquid samples 450 may be deposited.

The height 109 (shown in FIG. 1) of the illustrated embodiment is informed by common dimensions in the market for sample collection tubes (e.g. 15.3 mm×92 mm tubes from Sarstedt, and others). There is a wide market for sample collection tubes, and a market preference exists for collection devices of similar size and shape. Aligning the dimensions of the SafeCollect® device with the preferred dimensions accommodates automation with existing workflows and platforms. Other embodiments may be shorter or taller depending on customer preferences or future standardization of the sample collection market.

The diameter of the illustrated embodiment is defined by preferred dimensions in the market. Aligning the dimensions with common tube dimensions accommodates automation with existing workflows and platforms. Other embodiments may be wider or narrower based on customer requests or standardization of automation platforms.

In certain embodiments, the dimensions of the sealing surface 209 are informed by the material composition of the seal 200. In order to form a stable seal, a sufficiently large sealing surface is required to generate a stable bond between the sealing surface 209 and the seal 200. In some cases, the sealing surface 209 should be at least above 0.5 mm or range from about 0.5 to about 2.0 mm in thickness. In the illustrated embodiment, the sealing surface 209 is of about 1.3 mm in thickness. In instances wherein a small sealing surface (<0.5 mm) is considered, a weak seal may be formed between the surface 209 and the seal material 200. In such cases wherein a weak seal is formed, the sealed compartment 170 is prone to failure such as leakages at the seal. Leakages at the seal would be detrimental to the function of the seal 200 and sealed compartment 170: to sequester the liquid reagent in compartment 170 during transport or storage prior to introduction of a sample 450. A small sealing surface (<0.5 mm) also introduces difficulties in manufacturing: placing the seal on a small sealing surface requires that the placement of the seal must be precisely controlled and small deviations in alignment can result in seal failures. A larger sealing surface 209, coupled with a larger seal 200, allows for more tolerance in the manufacturing process improving the manufacturability of the device 10. As a result, the exemplary thickness of the sealing surface 209 is preferred for the device function and performance.

The dimensions of the sealing surface 209 is also a preferred feature in the formation of a seal between the sealing surface 209 and the seal 200. For a continuous seal along the sealing surface 209, the sealing surface 209 should have a flat surface profile to allow for maximum contact between the surface 209 and the seal 200. A warped, concave, convex, rough, wavy, or otherwise irregular sealing surface 209 would reduce the bond area formed between the surface 209 and the seal 200 which would negatively impact the integrity and stability of the seal. As a result, a manufacturing process and design that produces a flat, level sealing surface 209 is necessary for the performance of the device.

The configuration shown in the illustrated embodiment FIG. 1-3 can assist in the manufacturing processes for cylindrical tube 100, including for example, injection molding processes. In certain embodiments, the cylindrical tube 100 will be produced from thermoplastic resins via the manufacturing process of injection molding. In some embodiments, the device may be formed from polypropylene (PP) while in other embodiments the tube may be manufactured from polyethylene terephthalate (PET), high-density polyethylene (HDPE), or other suitable materials. In specific embodiments, the device may be manufactured from clear or transparent plastics. Alternate embodiments may utilize colored resins for decorative or descriptive purposes.

For specific embodiments, the preferred dimensions of both the sealing surface 209 (about 0.5 mm to about 2 mm) and the exterior (height 109 and diameter D) of the cylindrical tube 100, can introduce challenges during the injection molding process. In the injection molding process, a given part should have a uniform or nominal thickness for preferred injection mold design. In some cases, this nominal thickness can vary depending on the material and the part design. In the illustrated embodiment, a nominal thickness of about 1 mm is preferred for the molding of a polypropylene tube 100. In certain embodiments, the sealing surface 209 has a thickness of over 1 mm and is separate from the 1 mm nominal thickness of the exterior walls 103 and 104 of the cylindrical tube 100. Following a more traditional tube design, which has a flat exterior surface along the length of the tube, would result in a wall thickness of over 2 mm along the length of the inner wall portion 105 depicted in the illustrated embodiment and would create a variety of issues for the tube design.

In injection molding design, substantial increases or decreases in wall thickness (e.g. 10% variations in thickness for some plastics) results in a variety of issues for the design. In some cases, deviations from the nominal thickness can negatively impact the flow of the thermoplastic resin through the mold, which can result in short shots wherein the plastic is not sufficiently distribute throughout the mold and the parts are ejected with holes or incomplete features. In other cases, voids may form in the center of thick walled plastics. In yet other cases, deviations in wall thickness can result in non-uniform cooling of the plastic material. Non-uniform cooling can result in various degrees of warping, or sink marks, in the final part. Warping can result in non-uniform parts that deviate from the specified designs. In the case of cylindrical tube 100, warping could result in an uneven sealing surface 209, for instance. As described previously, an uneven or non-flat sealing surface would result in poor sealing and negatively impact performance of the part. Poor sealing would negatively impact overall performance of the products (e.g. loss of value due to discarded products, users exposed to the reagent, and loss of reagent during storage/transport such that insufficient reagent is available to appropriate stabilize biological samples).

In order to address problems related to wall thickness, the unique double wall design with inner wall 105 was devised to form the sealing surface 209 in the cylindrical tube 100. Here, double wall design refers to the use of inner wall 105, with tapered end 107, connected to outer walls 103 and 104 to form cylindrical tube 100 with sealing surface 209 on the internal cavity of the tube 100. Due to current dimensional restraints, and to prevent molding issues related to uneven wall thickness, a series of ribs 180 are found along the side of the tube. In preferred embodiments the ribs 180 or other structures provide for tubes without warping from the injection molding process and are needed due to constraints for preferred dimensions of the tube 100 and maintaining a 1 mm nominal thickness for the inner wall 105 of the tube. As previously discussed, the sealing surface 209 of the tube should be sufficient to form a strong seal with the seal 200. Additionally, the outer dimension of the tube is constrained by preferred dimensions in the industry. As a result, introducing the sealing surface 209 to the interior of a standard tube results in an overall wall thickness greater than twice the nominal thickness of the tube (1 mm). In the injection molding of thermoplastic resins such as polypropylene, drastic increases in wall thickness result in uneven cooling of the part, leading to a warping of the thick walled region. The ribbed features 180 allows for the tube to maintain a uniform wall thickness (1 mm), have an interior sealing surface 209 that is greater than 1 mm for forming a sufficient seal, and maintain an overall profile defined by the tube diameter D and height 109 preferred by the industry. Without the ribbed inner/outer wall design, various issues could arise including: a thin sealing surface insufficient for seal formation, a region of excess wall thickness resulting in warping of the tube and inconsistent part quality, non-uniform diameters along the length of the tube which could interfere with automated handling of the tube, or other issues that could negatively impact the manufacture and consistency of the tube.

In the illustrated embodiment, the exterior surface of the tube can be molded with a two slide process, in which the slides are pulled away from the part to eject tube 100 from the mold. In such embodiments, the slides are typically symmetrical and meet at a middle axis 183 of tube 100. Therefore, ribs 110, 120 and 130 are designed to allow the mold to separate from tube 100, and cannot include features (e.g. undercuts) that would inhibit separation of the mold. Additionally, ribs 110, 130 and 140 cannot be too thick or packed together without again introducing the issues related to excess wall thickness described above.

In certain embodiments, central ribs 110, first outer rib 130 and second outer rib 140 are configured to maximize the amount of material between the inner wall portion 105 and outer wall portion 103 of the tube 100. In automation and labeling processes utilizing tube 100, it is useful to have a continuous tube diameter. Accordingly, outer wall portion 103 has a diameter D that is consistent along the length of tube 100. In addition, ribs 110, 130 and 140 help maintain a continuous diameter without introducing problems of warping and other issues related to excess wall thickness described above.

In certain embodiments, each central rib 110 is tapered such that the thickness (e.g. the distance between each planar side 111) of each central rib 105 at curved end 112 is less than the thickness of each central rib 110 proximal to inner wall portion 105.

In view of the constraints above, the illustrated embodiment comprises an array of twelve ribs (i.e. ten central ribs 110, first outer rib 130 and second outer rib 140) used to maximize the surface area of the ribs on the exterior of the tube. First outer rib 130 and second outer rib 140 each have a larger arc of material in curved sides 132 and 142, respectively, as a result of the positioning of ribs 130 and 140 along axis 183 of a two slide mold process. The remaining ten central ribs 110 each have a curved end 112 which follows the arc of the outer tube diameter D. As a result, ribs 110, 130 and 140 maintain the circular exterior surface of tube 100.

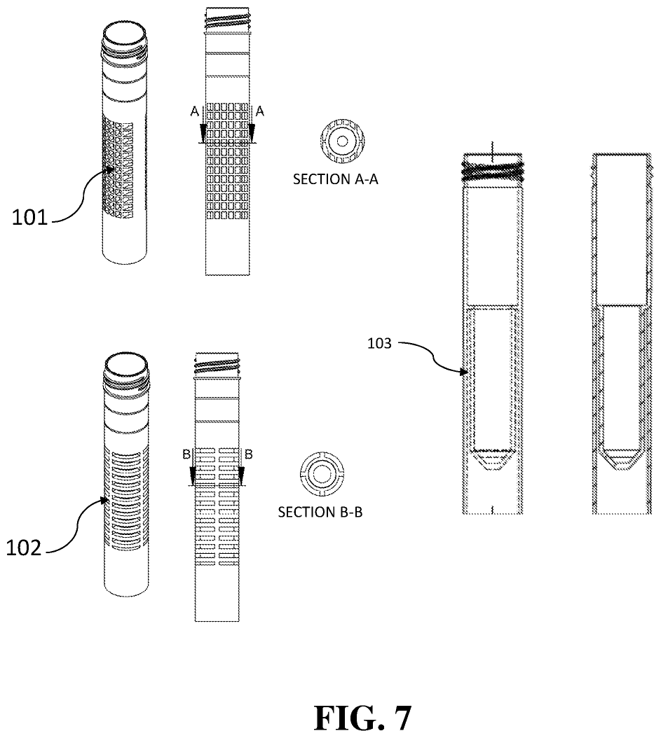

In the embodiment shown in FIGS. 1-3, ribs 110, 130 and 140 only span the tube along a single vertical direction (e.g. top to bottom). Alternate embodiments may also include lateral ribs forming a checkerboard type pattern or partial circumferential rings as show in FIG. 7. In such embodiments, the surface area of the ribs is further increased while still allowing sufficient cooling of plastic to prevent issues related to excess wall thickness. In other embodiments, it may be preferred that no vertical ribs are included, and only lateral ribs are added to maintain the surface of the tube.

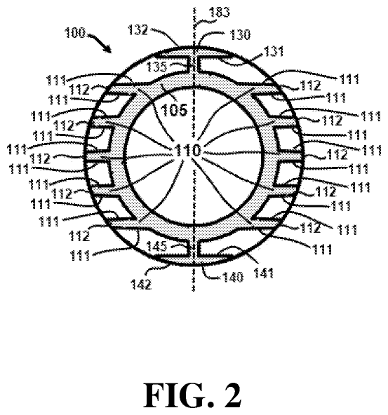

FIG. 7 shows an embodiment with lateral ribs laid over existing rib design detailed in FIG. 2 to form a checkerboard pattern 101. Another embodiment 102 has an alternate horizontal and vertical rib pattern. FIG. 7 also depicts another embodiment with narrow slots 103 in the bottom of the tube to maintain a consistent wall thickness. In some embodiments, a design incorporating narrow slots in the bottom of the tube may negatively impact the longevity of the mold, as narrow slots require thin metal inserts as part of the mold which are subject to excess damage and wear with use of the mold. In certain embodiments the pattern of ribs may have a preferred design or look for commercial acceptance of the specimen collection device added apart from functional aspects of the tube molding.

It should be noted that if the diameter D of tube 100 is sufficiently increased, alternate methods of forming sealing surface 209 are more viable. For instance narrow slots between an inner and outer wall (FIG. 7 embodiment 103) would be possible if the space between the inner and outer wall is sufficiently large (>1 mm).

It is noted that ribs increase the opacity of the material used in the manufacturing of tube 100, and therefore make it more difficult to visually see inside of the inner regions or compartments 160 and 170 of tube 100. In the illustrated embodiment, users can clearly see liquid inside of the sealed compartment 170 of tube 100 through the regions of inner wall 105 between ribs 110, 130 and 140. In alternate embodiments with lateral ribs, the ribs may inhibit clear visibility of the liquid, but in such embodiments the liquid visibility may or may not be a desired feature.

In other embodiments the ribs may be used to provide for a preferred design or look of the tube in addition to their function for preventing warping due to wall thickness and overall tube dimensions in the molding process. In some embodiments, the ribs provide for improved gripping when users handle the tube 100. The necessity of a sufficient sealing surface introduces the need for the inner wall portion 105 and the outer wall portions 103, 104 to avoid an excessively thick-walled section of the tube 100. Sealed compartment 170 is separated from compartment 160 by pierceable seal 200. In the embodiment shown, compartment 160 can be accessed by removing cap 500 from first end 101 of tube 100.

The illustrated design incorporating an inner wall 105 and outer wall 103, 104 and ribs 110, 130, 135, 140, and 145 to produce a sealing surface 209 onto which a stable seal can be deposited is not presently observed outside of the embodiments described herein. The varied design constraints, including a sufficiently larger sealing surface 209, a uniform tube 100 diameter D and height 109 that conforms to preferred dimensions in the industry, and a part that can be injection molded efficiently necessitated a unique approach to design of the tube 100. Without the novel double wall tube design depicted in the illustrated embodiments, the tube 100 could not be produced without sacrificing desired features (e.g. reducing the size of the sealing surface 209 or increasing the thickness of the part along the inner wall 105 which would result in warping or deformations during the injection molding process).

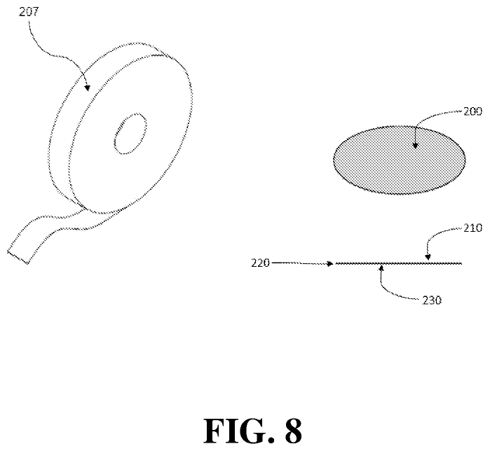

Referring now to FIG. 8, pierceable seal 200 is comprised of a material that is a pierceable foil seal or other suitable sealing material. In the illustrated embodiment, the sealing material is to prevent the release of the liquid reagent contained in sealed compartment 170. The seal also creates a barrier between sealed compartment 170 and the upper compartment 160 of tube 100. To create a stable seal between the sealed compartment 170 and upper compartment 160, the material is to be of sufficient strength to remain intact prior to use. The seal cannot fail during storage or transport, including rough shipping conditions, drops from elevated heights, or vigorous shaking to fully mix the sample with the reagent and ensure proper stabilization of the sample. In some specific embodiments, the seal 200 should remain intact when the tube 100 is dropped from heights of over 10 ft. In other embodiments, the seal should remain intact during reduced pressure experienced during air travel (e.g. a vacuum of 11 inHg, or up to 27 inHg). While remaining stable during shipping or transport, the seal must also be pierceable with a swab or other blunt instrument.

FIG. 8 shows the seal 200 that is applied to the interior of the tube 100. In some embodiments, the seal 200 is initially supplied as a reel 207, which is then punched to form the circular seals delivered to the interior of the tube 100 during the manufacturing process. In some embodiments the seal 200 is a pierceable foil seal 15-50 micrometers in thickness. In other embodiments, a seal with thickness greater than 50 micrometers may be preferred. The material is of sufficient strength to remain intact prior to use, but also pierceable by a swab or other blunt instrument without significant force. In the current embodiment the seal 200 is composed of 3 layers (e.g. Proforma IntegraFoil PP Pierce, Ballerstaedt BaCo Seal Unicoat ECO 2.1, or other materials). In other embodiments the seal 200 may be composed of 1, 2, 3, or more layers.

In the embodiment shown in FIG. 8, a top layer 210 of the seal 200 is a lacquer to protect the foil from oxidation. In one embodiment, this lacquer is composed of nitrocellulose. In other embodiments, the lacquer may be replaced with another material or polymer. The lacquer is to be inert with samples or stabilizing reagent. In the illustrated embodiment, a middle layer 220 of the seal 200 is an aluminum foil. The aluminum provides structural support to the seal while being thin enough to be pierced. While aluminum is the most prevalent metal used in sealing materials, other embodiments with alternate metal materials (e.g. gold, tin, nickel, and others) may be viable. The illustrated embodiment includes a bottom layer 230 that is a heat sealing lacquer that can seal to the tube 100 sealing surface 209 (composed of polypropylene in one embodiment). In specific embodiments, this heat sealing lacquer is composed of a thermoplastic polymer such as PP, PET, PE or other thermoplastic. In alternate embodiments, the sealing material 200 may be composed of more than 3 layers of material. For instance, an additional layer of sealing agent may be included between the heat seal lacquer 230 and the aluminum foil 220. In other cases, additional layers of protective lacquer may be applied at various levels of the sealing material 200.

In some cases, the stabilization reagent contained in sealed compartment 170 maybe be corrosive to metals including aluminum. In such cases, a protective lacquer layer 210 and sealing lacquer layer 230 serve to protect the aluminum layer 220 from corrosion.

In the illustrated embodiment, the sealing process is performed by an induction sealer. Other embodiments may use heat sealing, or other methods. The sealant is to be inert with the sample and stabilizing reagent. The sealant is to remain intact during shipping, storage, and transport and resistant to degradation by the stabilizing reagent. Individual seals 200 can be cut or punched from a foil reel of material 207 . . . . In any embodiment, the sealing layer must maintain a sufficient seal when exposed to the stabilizing reagent at various temperatures and pressures experienced during shipping. Furthermore, the seal should remain intact for extended periods of time to ensure the device will have a sufficiently long shelf life during storage or transport prior to use in collecting a sample. The sealant should also be inert with the stabilization reagent and the biological sample.

In one embodiment, the seal 200 is applied to the tube 100 via induction sealing. In the induction sealing process, an electric current is used to generate heat in a conductive (i.e. metal) material which melts the thermoplastic adhesive layer 230, allowing it to bond to the sealing surface 209 of the tube 100. In some manufacturing methods, significant pressure is applied to seal material 200 while the inductive current is applied, further improving the strength of the seal. In the inductive sealing process, the aluminum foil layer 220 of the seal 200 is necessary to generate the heat when an inductive current is applied to the seal. Without the aluminum foil layer 220 (or similar conductive material), the use of induction sealing would not be a viable manufacturing method.

Other embodiments may use alternative methods to apply the seal to the tube. Such alternative methods include, but are not limited to, heat sealing and ultrasonic welding. In such cases, the use of foil seals may be discarded in favor of polymer seals, such a thermoplastic film without a foil or metal layer 220. Thermoplastic films could include, but are not limited to, PP, PET, and PE as described for the heat sealing lacquer 230 in the illustrated embodiment. In some embodiments, thermoplastic films may be composed to just one layer, but may be composed of more than one layer. Thermoplastic films could be adhered to sealing surface 209 via heat sealing, ultrasonic welding, or other methods without the need for a metal layer 220. The preferred composition of the sealing material 200 would be dependent on manufacturability and performance, as any foil or thermoplastic seal would still need to maintain the functional features of the internal seal described previously.

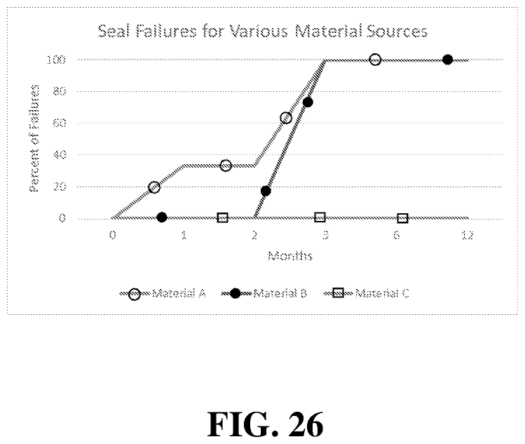

FIG. 26 demonstrates that only a subset of available seal materials meet the performance criteria required for a SafeCollect® device. In this experiment, 3 different sealing materials (Material A, B, or C) were used to produce a sealed tube containing a stabilization reagent. The tubes were then subjected to accelerated aging conditions to evaluate the integrity of the seals over time when exposed to the stabilization reagent. At the 3 month timepoint 100% of devices sealed with Material A and Material B had failed, meaning that seal was compromised and the reagent had leaked out of the sealed chamber. Material C, in this experiment Proforma IntegraFoil PP Pierce, successfully maintained a seal over a long period of time. This experiment highlights the uniqueness of the invention, as only a subset of components and materials are viable to produce the SafeCollect® device.

FIG. 4 illustrates in panels [A], [B] and [C] one embodiment of the invention for the collection of saliva samples. The device includes a collection tube 100, a circular seal 200, a piercing cap 300, and a funnel 400. The tube is provided with the foil seal applied and contains a stabilization reagent.

In some embodiments, the tube 100 is provided to the users with the funnel 400 attached as shown in FIG. 4C. After deposition of a sample 450, the funnel 400 can be removed and the piercing cap 300 can be screwed onto the tube 100 via corresponding threads on the cap and tube, piercing the foil seal 200 and allowing the stabilization reagent to mix with the sample. The capped tube shown in FIG. 4B can be shipped or stored while stabilizing the biological contents of the sample.

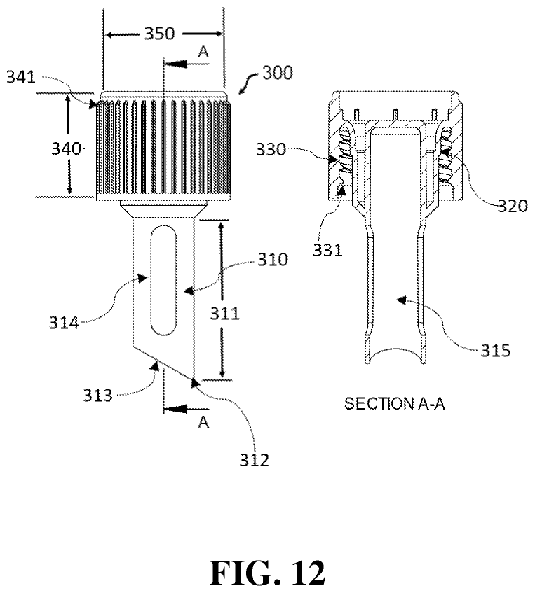

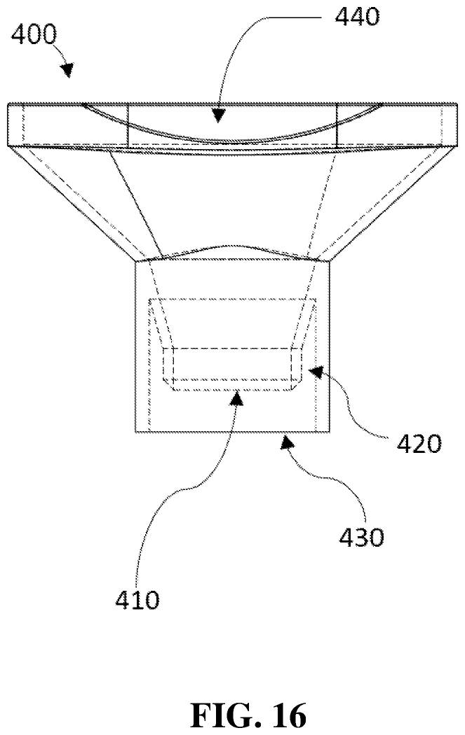

FIG. 4, FIG. 6, FIG. 8, FIG. 12, and FIG. 16 illustrate relevant components of a saliva collection device. In an embodiment shown in FIG. 4, a user may remove cap 500 from first end 101 and then couple funnel 400 to first end 101 of tube 100. It is understood that in certain embodiments, tube 100 may be provided to a user without cap 500. Seal 200 contains any liquid contents of tube 100 without a cap coupled to first end 101. Certain embodiments may also comprise a different configuration of cap, including for example, a piercing cap 300. Funnel 400 is configured to assist in depositing a sample 450 (including, for example, saliva, sputum, or other liquid or liquified samples) into compartment 160. Pierceable seal 200 prevents the sample 450 from entering sealed compartment 170. In certain embodiments, sealed compartment 170 may comprise one or more reagents, including for example, stabilizing reagents (i.e., Specimen or Viral Transport Media either commercially available or custom made for applications). After sample 450 is placed in compartment 160, piercing cap 300 can be coupled to first end 101 of tube 100 if it is desirable to mix sample 450 with the contents of sealed compartment 170. As piercing cap 300 is coupled to first end 101, pierceable seal 200 is ruptured or pierced by a unique piercing element 310 (sometimes referred to herein as the Sure Punch piercing element) included on the cap, allowing sample 450 to mix efficiently with the contents of sealed compartment 170.

FIG. 16 illustrates a funnel 400 that can be used to collect a sample, including for example, saliva. The funnel engages with the opening 110 of the tube 100 to allow for users to deposit saliva (or other liquids) into the upper compartment 160 of the tube 100. An outlet 410 of the funnel is wide without surfaces parallel to the ground or base of the funnel. Due to the viscosity of some samples, narrow openings or some angles can result in clogging of the funnel and prevent material from flowing into the opening 110 of the tube 100. A fitting 420 around outlet 410 of funnel 400 fits snuggly into the opening 110 of the tube 100, forming a press fit between the funnel and the tube. The press fit holds the funnel 400 in place during use with the tube 100. Users can easily remove the funnel 400 from the tube 100 with a light twist. Other embodiments may include threading such that the funnel 400 can be screwed onto the tube 100 via the threading 115. Still other embodiments may use some other method of mechanical coupling to attach the funnel 400 to the tube 100. A diameter 430 of the funnel outlet 410 is wide enough to fit over the first end 101 of the tube 100 and threading 115. Funnel 400 has an inlet or opening 440 to accommodate convenient and comfortable collection of saliva or other liquids 450. Opening 440 is sufficiently wide to prevent saliva from missing the tube when deposited. The funnel 400 presented in FIG. 16 is one embodiment of the funnel design, alternate funnels may be developed to suit unique functional or decorative needs.

When a sample 450 is added to the SafeCollect® tube 100, the seal 200 remains intact and the sample is held in the upper compartment 160 of tube 100. The seal 200 will not be pierced until the piercing cap 300 with the Sure Punch piercing element 310 is fully engaged and screwed onto the tube 100 via threading 115. In certain embodiments, liquid reagents located in sealed compartment 170 should not come into contact with the user supplying a sample. By piercing the seal 200 only after the tube 100 is closed with cap 300, the user is not exposed to an reagents during use of the device. This improves the safety and functionality of the device.

Conventional tube designs would not be able to sequester harmful reagents into a seal compartment, and the unique SafeCollect® design allows for the novel safety feature of a sealed compartment 170 to be possible. Additionally, the seal could not be pierced with a conventional cap design and the piercing cap 300, with a novel Sure Punch piercing element 310 incorporated into the design allows users to cap the tube 100 and pierce seal 200 simultaneously without any exposure to the reagents. This design is especially important for at home collection scenarios, where untrained individuals are collecting their own saliva or liquid sample without assistance from a trained technician. The SafeCollect® design prevents users from interacting with the reagent enclosed in sealed compartment 170 until the tube is capped and ready for shipment, even in the event of dropping the tube prior to capping.

Referring now to FIG. 12 for further description of the piercing cap 300 with Sure Punch piercing element 310, which can be placed onto the tube after a sample 450 has been provided and the funnel 400 has been removed from the tube 100. The Sure Punch piercing element is design to efficiently open foil seal 200 to allow for mixing of sample 400 in upper compartment 160 with the desired reagent in sealed compartment 170. In certain embodiments, to achieve preferred mixing, the Sure Punch piercing element 310 is to generate a wide opening the seal 200 at the opening 106 of the sealed compartment 170. In preferred embodiments, the seal should create a circular punch of foil that is pushed to the side of the tube to allow mixing between upper compartment 160 and sealed compartment 170. Additionally, in preferred embodiments, the foil should remain attached to the seal surface 209 by an arc of approximately 20-45 degrees; if the foil is fully punched out it can result in a free floating foil disc which can interfere with mixing and inhibit removal of liquid from tube 100. Given the constraints for the piercing cap 300 described, a novel design is presented for a cap to pierce an internal seal 200 in a tube 100 during the capping process.

As seen in FIG. 12, piercing cap 300 includes a novel piercing element (denoted Sure Punch piercing element) 310 extending from the center of the cap 300. In preferred embodiments, Sure Punch piercing element 310 has a length 311 that is sufficient to pierce seal 200 after being completely coupled to tube 100 (e.g. fully threaded onto end 101 via threads 115). In certain embodiments, when piercing cap 300 is placed onto tube 100, but not fully engaged with the threads 115, Sure Punch piercing element 310 is not long enough to pierce seal 200. In certain embodiments, Sure Punch piercing element 310 comprises an end 312 with an angled surface 313 that form an edge with a shallow angle. As cap 300 is fully coupled to end 101 (e.g. completely engaged with the threads 115), end 312 pierces or cuts into pierceable seal 200 and continues to cut pierceable seal 200 as cap 300 is threaded onto threads 115. In certain embodiments, Sure Punch piercing element 310 has a fully circular cross-section and forms a circular cutout of pierceable seal 200 that is pushed aside from the sealing surface 209 by the circular shape of Sure Punch piercing element 310. In other embodiments, the Sure Punch piercing element 310 has a semi-circular cross-section. This piercing action allows for the barrier between the upper compartment 160 and lower sealed compartment 170 of tube 100 to be fully opened. This further allows efficient mixing between the contents in the upper and lower compartments of tube 100.

In certain embodiments, apertures 314 are formed into the sides of the Sure Punch piercing element 310 to further facilitate efficient mixing between the upper compartment 160 and lower sealed compartment of the tube 100. When cap 300 is engaged, tube 100 can be inverted and/or shaken to mix the contents of tube 100 vigorously. In certain embodiments, Sure Punch piercing element 310 prevents pierceable seal 200 from moving during inversion, which could inhibit mixing between the upper and lower portions of the tube. In the embodiment shown, Sure Punch piercing element 310 comprises an interior portion 315 that is hollow in order to limit the volume of Sure Punch piercing element 310 and prevent Sure Punch piercing element 310 from pushing out sample 450 from compartment 160. Other embodiments not illustrated herein may deviate from the Sure Punch piercing element 310 described, and may have a different piercing element that pierces the seal prior to thread engagement, or at a different number of thread rotations.

As shown in the cross section in FIG. 12, in certain embodiments an upper portion of piercing element 310 includes a rounded ledge 320 that presses against the inner walls of the tube 100 as the cap is threaded onto tube 100. The ledge 320 engages with tube 100 as the cap is screwed on forming an interference fit along the interior wall of tube 100. The interference fit prevents liquid from leaking out of the tube before pierceable seal 200 is pierced. Accordingly, the user will not be exposed to the stabilizing reagent or other contents of compartment 170 unless the cap is unscrewed from tube 100. In other embodiments, a variety of features could be considered to achieve the same function as ledge 320 including but not limited to, an o-ring incorporated into the cap, a molded ring of plastic in place of ledge 320, or other features to form a fit and prevent leaking from the tube 100 when the cap 300 is engaged with opening 101 of tube 100.

In the illustrated embodiment, cap 300 is manufactured via injection molding. In preferred embodiments, cap 300 is made from high-density polyethylene (HDPE). In alternate embodiments, the cap 30 may be composed of alternate thermoplastic polymers such as PP or PET. In specific embodiments, the cap 300 may be transparent while in others it may be colored. The look and color of the cap 300 can be adjusted to fit a particular decorative need or demand.

The injection molding of threaded parts can be achieved in a variety of ways. Generally threading introduces undercuts to the part, meaning that the plastic interferes with ejection of the part from the mold. In the case of threaded parts, this can be resolved with a few different methods, two of which are unscrew mold designs and strip threading or “popoff” mold designs. Unscrew mold designs are molds designed to unscrew out of the threaded region during ejection. With unscrew molding, a variety of thread profiles are viable, as long as they are continuous in nature. The metal part of the mold simply follows the thread profile out of the part and the part is ejected. In the “popoff” method, the part is forcefully ejected from the mold while the plastic is hot and the threads are stripped out of the mold. “Popoff” ejection for threaded parts can damage the threaded features if they are not designed properly. For efficient ejection in a “popoff” mold, threads should have an angular profile angled in the direction that the metal mold is moved. In this way the mold can be removed from the part without altering the tread profile. Given the variety of methods for injection molding a threaded part, different embodiments of the design may alter the thread profile to accommodate unscrew molding, “popoff” molding, or other methods.

The threading 330 of cap 300 is designed to engage with threading 115 of the tube 100. In the illustrated embodiment, the threading profile 331 is designed for a ‘popoff’ injection molding process. In certain embodiments, an unscrew mold design may be preferred. In other embodiments, the Sure Punch piercing element feature of the cap 310 may limit the viability an unscrew mold design. In particular embodiments, the threading profile 331 is angled so that the parts can be ejected from the mold while the plastic is still hot.

In specific embodiments, the threads 330 span a length of the cap 300 to allow for enough rotation to 1: engage the sealing ledge 320 and 2: engage the Sure Punch piercing element 310 to fully pierce the seal 200. Other embodiments may alter the thread rotations to adjust when piercing occurs when engaging the cap 300.

In specific embodiments, the height 340 of cap 300 is optimized for automated decapping on existing platforms. Grips 341 along the side of cap 300 allow for users to tightly screw cap 300 onto tube 100 and allow for piercing of pierceable seal 200. Other embodiments may have different heights for decorative or functional purposes.

Diameter 350 of cap 300 is informed by the diameter D of tube 100. The diameter was optimized for automated decapping on existing platforms. Other embodiments may have different diameters for decorative or functional purposes.

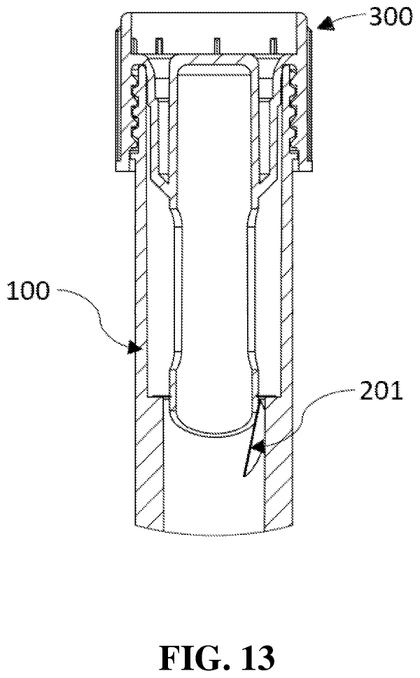

FIG. 13 illustrates a cross section of tube 100 in which piercing cap 300 has pierced the seal 200 with circular Sure Punch piercing element 310 to create a pierced seal 201. As shown in this embodiment, pierced seal 201 is directed to the side of tube 100 to allow mixing of the contents previously separated by the seal. The unique features of the Sure Punch piercing element 310 (length 311, end 312, angled surface 313) all play an important role in the function of the Sure Punch piercing element. Adjustments to the features of the Sure Punch piercing element 310 can result in inadequate or inefficient piercing of seal 200, and negatively impact performance of the SafeCollect® design as a whole. For instance, alterations to the length 311 could result in no piercing of seal 200, or piercing that occurs before the cap 300 is engaged with threading 115, which could expose the user to the reagent contained in sealed compartment 170. Other changes could result in the foil being completely torn, forming a free-floating disc which interferes with mixing and removal of liquid from the device. Alterations to the end 312 and angled surface 313 can result in a piercing element that does not form a circular punch out, and instead just creates cuts in the foil seal 200, again inhibiting mixing between the upper compartment 160 and lower sealed compartments 170 of the tube 100. As demonstrated, the design of the Sure Punch piercing element 310 is non-trivial requires those skilled in the art to design, evaluate, and manufacture. Alternate embodiments of the Sure Punch piercing element 310 may adjust the parameters of the piercing element (length 311, end 312, or angled surface 313), but only a subset of adjustments conserve the performance and function of the Sure Punch piercing element described by the illustrated embodiment.

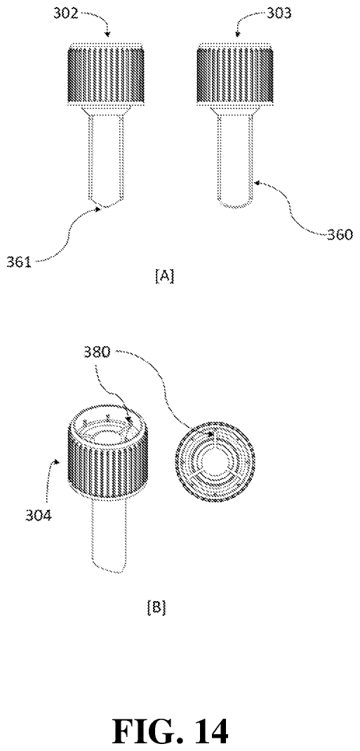

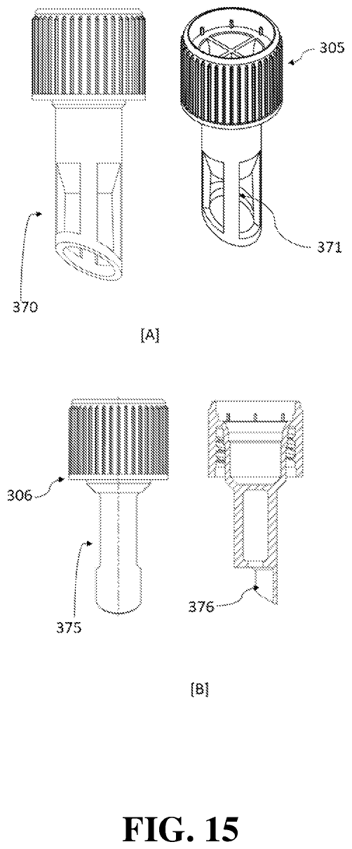

FIGS. 14 and 15 illustrates an alternate cap designs suited towards specific applications for obtaining defined sample volumes. Some embodiments may alter the piercing element to accommodate different sample types.

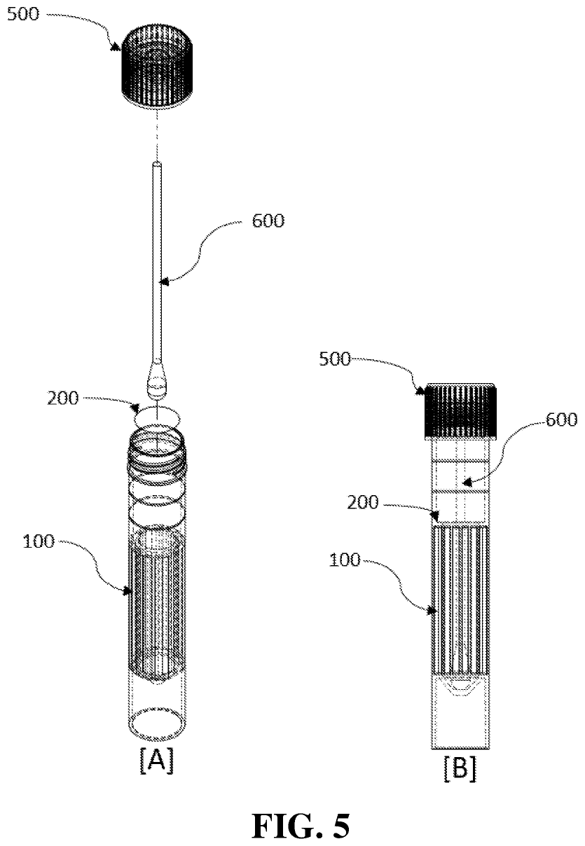

FIG. 5 illustrates in panels [A] and [B] a second embodiment of the invention for the collection of swab samples. The device in FIG. 5 includes a collection tube 100, a circular seal 200, a swab cap 500, and a swab 600. The tube 100 is provided with the circular seal 200 applied and contains a stabilization reagent in sealed compartment 170. The swab 600 is deposited into the tube and serves to pierce the seal, allowing the tip of the swab 610 to be submerged into the stabilization reagent. The tube 100 can be capped with the swab cap 500 and shipped or stored as a capped tube shown in FIG. 5 panel [B]. The swab cap 500 will catch the swab 600 and retain the swab 600 during the decapping process. When preferred, the swab cap may be exchanged for a standard cap design that does not capture the swab 600. In such cases, the swab 600 remains in the tube 100 when decapped.

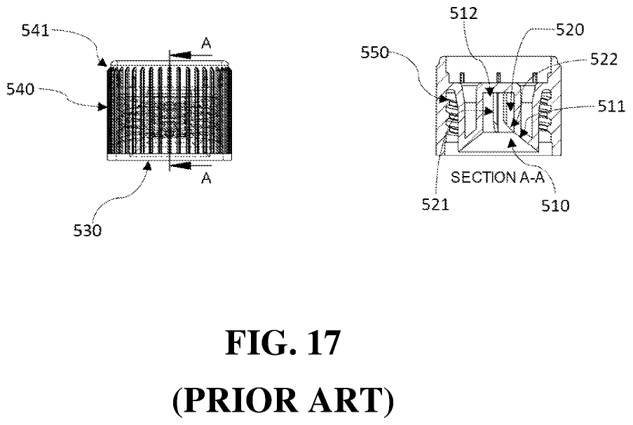

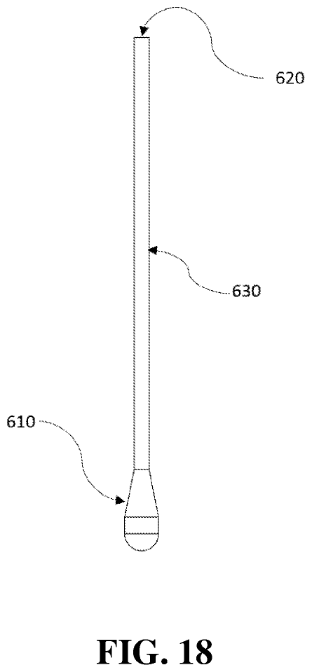

FIG. 5, FIG. 6, FIG. 17 and FIG. 18 illustrate components of an embodiment with a swab and tube with a swab head 610 which generally collects the sample and pierces the foil seal 200 in this embodiment of tube 100. The swab head 610 may vary in other embodiments to accommodate different sample types or collection methods. The swab 600 also comprises a swab end 620 which will be captured by the swab cap 500. Generally, the swab end 620 is produced from a breakpoint in a longer swab. In some embodiments, the swab may not include a breakpoint and is an appropriate length when used. The swab end fits into the center channel 520 (shown in FIG. 17) of the swab cap where it is held in place. A length 630 of the swab is to be large enough to go from bottom 107 of tube 100 to the center channel 520 of the cap. Certain embodiments are for a swab length common in the field, defined as 80 mm in certain embodiments. Specific embodiments may adjust the swab length for different applications or designs.

Exemplary embodiments disclosed herein provide a swab-based collection system that partitions a stabilization reagent in an interior sealed compartment 170 of collection tube 100. Existing swab collection systems have a reagent filled tube that is provided capped, and then uncapped to place the swab. In this process, the user can spill the reagent, as the reagent is not sequestered in any way after the cap has been removed. In existing swab collection systems, the user may accidentally inject the reagent, dip the swab in the reagent prior to collecting their sample, or otherwise come in contact with the reagent contained in the tube. A variety of stabilization reagents used in the collection of swab samples can be harmful to human health when ingested or touched. Some of the problems associated with harmful reagents can be alleviated when samples are collected by trained medical personnel or technicians, learned in the sample collection process and knowledgeable of how to collect swab samples properly. However, in the context of self collection in home setting, the general population is at significant risk for collecting a swab sample improperly and exposing themselves to harmful reagents used in the field of sample collection and stabilization.

With embodiments of the SafeCollect® system displayed in FIG. 5, FIG. 6, FIG. 17, and FIG. 18, the reagent cannot be spilled or, more importantly, come into contact or be ingested by an individual donating the sample, until the swab sample has been inserted into the tube and has pierced the seal 200 to be submerged in the reagent contained in sealed compartment 170. This makes the SafeCollect®™ design a preferred safe design appropriate for home collection as compared to existing swab collection methodologies that are prone to spillage or user interaction with a reagent. Users will not be able to ingest or spill harmful reagents prior to collecting their swab and inserting it into the tube after which the tube is capped. No known design for the collection of biological samples with a swab presently incorporates an inner sealed chamber in a collection tube to protect users from exposure to stabilization reagents. The use of the SafeCollect® design for the collection of swab or swab-type biological samples is a unique and device for collecting the samples.

Referring back to the tube design depicted in FIG. 6, certain embodiments, include an array of ribs 180 present on the exterior of the tube 100. The generation of the sealing surface 209 results in thick walls compared to the nominal thickness of the tube 100, which can result in problems during the injection molding process as described previously. The use of the inner wall 105 design described previously was developed to address the problems related to thick walls while maintaining a consistent external dimension. Future embodiments may use a different ribbing pattern, or eliminate the ribs. In certain embodiments, bottom 107 of the tube is deep enough for swab dimensions common in the market (i.e. 80 mm breakpoint swabs). The tube is compatible with 80 mm breakpoint swabs currently available from various suppliers. The bottom of the tube 190 is the same length as the swab 600, to facilitate common breakpoint swab lengths.

Other embodiments may adjust the tube depth to accommodate other collection methods. Other swab sizes and breakpoints do exist on the market (e.g. 16-27 mm breakpoints are available from different suppliers). To accommodate alternate swab designs and dimensions, alternative tube embodiments of different dimensions may be developed. In other embodiments, the swab can still be used to pierce the seal 200 and dropped into the sealed compartment 170 of the tube 100 to be submerged in the stabilization reagent. In yet other embodiments, the swab may be broken off in the upper compartment 160 of the tube 100 and a piercing cap 300 with a Sure Punch piercing element 310 design could be used to cap the tube with the swab which would open the seal 200 and allow the swab to drop into the sealed compartment 170 of the tube 100 to submerge the swab collector. Capture of the swab end 620 by a cap 500 is but one embodiment of a swab 600 used with a SafeCollect® tube 100. Other embodiments may prefer to use a cap that does not capture the swab end 620.

As described previously, a seal 200, is applied to the sealing surface 209 producing an upper compartment 160 and lower sealed compartment 170 separated by the seal 200. In one embodiment, the lower sealed compartment 170 of the tube 100 is filled with stabilizing reagent. The lower sealed compartment 170 of the tube 100 can hold over 2 ml of liquid in one embodiment. In other embodiments, 500 ul, 1 ml or 1.5 ml other volumes may be stored in sealed compartment 170 of the tube 100. To produce sealing surface 209, an array of ribs 180 are positioned around the exterior of the inner wall 105 of the tube 100. Due to the constraints of the external tube dimensions, and the need for a sufficient area for the sealing surface 209, thick walls larger than the nominal thickness of the tube may occur. Thick walls can result in poor performance when injection molding the part, as it can result in uneven cooling of the plastic. To overcome the problematic warping issues related to thick walls, the walls were replaced with thin ribs which allow the part to cool in a uniform fashion while maintaining the external dimensions of the tube. Other embodiments may adjust the internal or external dimensions such that ribs are not needed. Alternatively, some embodiments may alter the design of the rib pattern.

FIG. 7 illustrates alternative embodiments of the tube design. Tube 101 and 102 have different ribbing patterns. Tube 103 has narrow slots in the bottom of the tube to maintain the nominal thickness of the tube walls while still forming the sealing surface 209 for placing the seal 200

In applications for swab collection, considerations of the seal 200 thickness and pierceability are required. In some embodiments of swab 600, the swab head 610 may be blunt or pliable. The seal 200 in SafeCollect® tube 100 should be pierceable with minimal force while still remaining intact during storage and transport. In some specific embodiments, the seal may be pierced with a swab with only 1.5 lbs of force. In other embodiments, 1-5 lbs of force may be required to pierce the seal 200 with the swab 600. For at home sample collection, those with disabilities or elderly users may be the user of a device to collect a swab sample and unable to contribute higher amounts of force to the swab 600 when piercing the seal 200. Only a subset of seal materials available for seal 200 are sufficiently thin to be pierced by a blunt swab 600 with 1.5 lbs of force. Some seal materials evaluated for use as seal 200 have thicker heat seal lacquer layer 230, foil layer 220, or protective lacquer layer 210 and were found to require a significant amount of force to be pierced by a swab 600 (e.g. Thermal seal 4ti-0591 from Azenta Life Sciences). Such seal materials were excluded for the manufacture of SafeCollect® device, despite forming a good seal and meeting other parameters for seal material described previously.

FIG. 17 illustrates a prior art cap design depicted in EP0366826A1 (expired). When the cap 500 is screwed onto the tube 100 containing a swab 600, the end of the swab 620 will be guided into the center 510 of the cap 500 and lodged into a circular array of fins 520. When the cap 500 is removed, the swab 600 is carried out of the tube 100 with the cap 500. The center 510 of the cap 500 inserts into the tube 100. An angled feature 511 produces a self-centering effect to guide the swab 600 into the center channel 512 of the cap 500. An array of fins 520 are located in the center channel 512 of the cap 500. As the end of the swab 620 is pressed into the channel 512, these fins 520 create a press fit with the swab 600 that holds the swab in place. The fins 520 are thin with a pointed tip 521 to accommodate common swab diameters. The fins 520 are angled at the bottom 522 to aid in centering the swab 600 as the press-fit is formed between the swab 600 and the cap 500. The diameter 530 of the cap 500 is designed for the diameter of the tube 100. The diameter 530 was optimized for automated decapping on existing platforms. Other embodiments may have different diameters. The height 540 of the cap 500 is optimized for automated decapping on existing platforms. Grips 541 along the side of the cap 500 allow for users to tightly screw the cap onto the tube 100. Other embodiments may have different heights. The threading 550 of the cap 500 is designed to engage with the threads 115 of the tube 100. In the current embodiment, the cap design allows for threading 550 that is molded via an unscrewing mold. Other embodiments may alter the threading profile to allow for pop-off molding. In specific embodiments, a sealing ledge (320 as observed in piercing cap 300) may be incorporated into the design to improve the fit between cap 500 and tube 100, minimizing leakage. Alternatives to a sealing ledge include, but are not limited to, an o-ring, a molded ring, and other features.

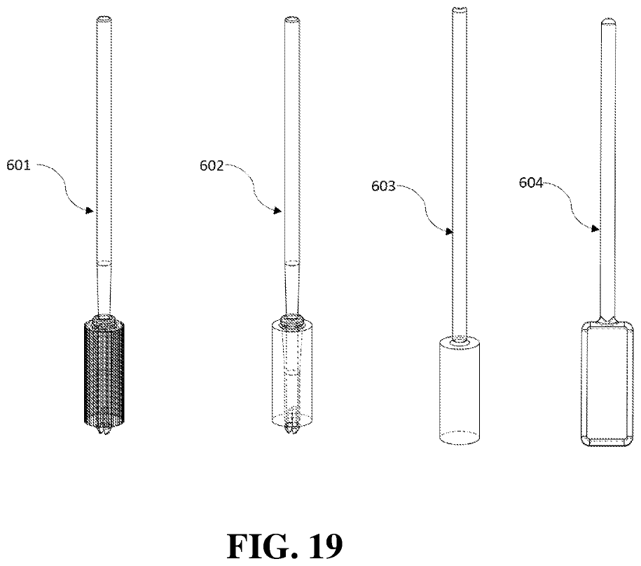

FIG. 19 illustrates a variety of designs of swabs 601, 602, 603 and 604 proposed for use with the tube 100. These include adhesive rollers for the collection of skin or other biological surfaces or samples or biofilms, or other substances, from environmental surfaces. Swab designs compatible with the tube are not limited to these embodiments. Swab 601 is an embodiment with a ridged roller design. In one embodiment, the roller would be coated in an adhesive to allow for the collection of skin samples, or other surface sample. Swab 602 is an embodiment with a smooth roller design. In one embodiment, the roller would be coated with an adhesive to allow for the collection of skin samples, or other surface samples. Swab 603 is an embodiment with a solid roller design. In one embodiment, the roller would be coated with an adhesive to allow for the collection of skin samples, or other surface samples. Swab 604 is an embodiment with a flat paddle design. In one embodiment, the roller would be coated with an adhesive to allow for the collection of skin samples, or other surface samples.

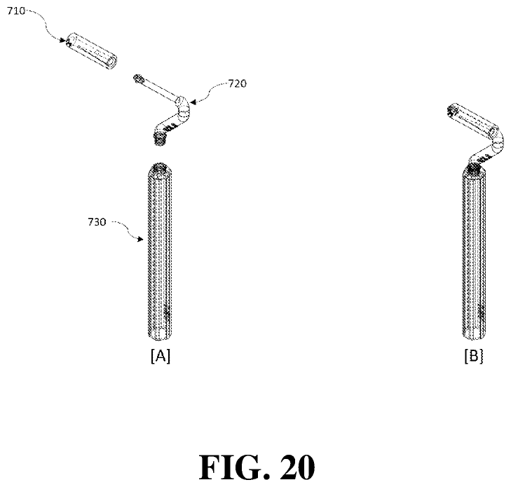

FIG. 20 illustrates an adhesive roller 710 for the collection of the sample. In some embodiments, the roller is coated with a liquid adhesive via spray or other method. In other embodiments, the adhesive is applied as a tape. The roller has a central hole that is tapered from a large diameter down to a small diameter for placement onto the spoke. A roller spoke 720 is also shown onto which the roller is placed. The spoke is notched at one end such that the tip collapses to a smaller diameter as the roller is pushed on, then expands back to hold the roller in place. In some embodiments, the spoke may also have threading on the other end to connect with the handle. Some embodiments may not have threading. These drawings represent but one embodiment of an adhesive collector device. This embodiment also includes a roller handle 730, a handle for comfortably using the roller on the face or other areas. The roller connects to the spoke via threading.

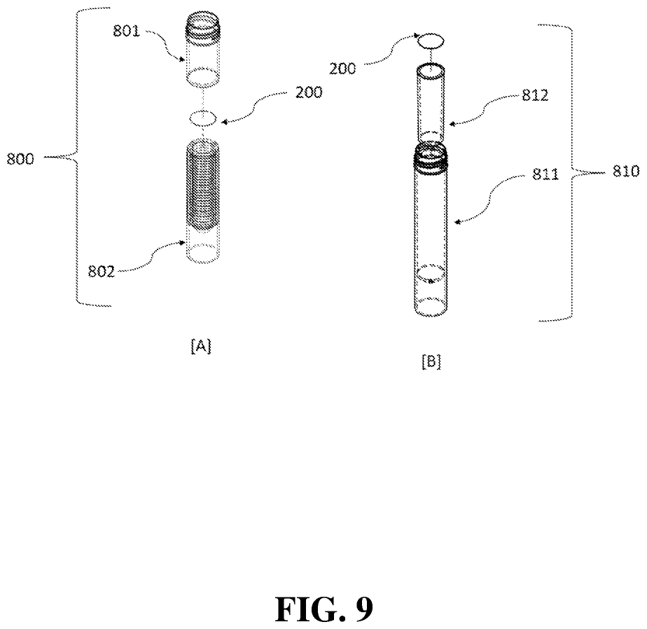

The embodiments of the SafeCollect® device illustrated in FIGS. 1-6 utilize but one method of manufacturing an internally sealed sample collection device wherein the device is manufactured as a single plastic part and the seal 200 is delivered to the interior of the device. FIG. 9 illustrates in panels [A] and [B] alternative methods for manufacturing the internal sequestration feature of the SafeCollect® sample collection device. For instance, in one embodiment show in FIG. 9A the device 800 is composed of two separate plastic parts, a top portion 801 and a bottom portion 802. The bottom portion, 802, could be filled with a liquid reagent and sealed with a seal 200 followed by a joining of the top portion 801 and bottom portion 802 of the device into a single part to produce the internal seal. Joining of the top 801 and bottom 802 portions of the device could be accomplished by various means, including but not limited to ultrasonic welding, laser welding, adhesives, press fit, threaded fit, or other methods.

In another embodiment (FIG. 9B) of the device 810 there may be a capsule 812 which is filled with a reagent and sealed with a seal 200. The capsule 812 is composed of a cylindrical tube composed of an open end and a closed end. The capsule 812 is filled with a reagent and sealed at the open end by seal 200 via induction sealing or the other methods described previously. The filled capsule 812 could then be inserted into an outer shell 811. Outer shell 811 would comprise a cylindrical tube composed of an open end and a closed end. In some embodiments, outer shell 811 may be a standard commercially available tube or the SafeCollect® tube design described. The capsule 812 would be inserted into the open end of the shell 811 and joined with the shell via a variety of methods including but not limited to ultrasonic welding, laser welding, adhesives, press fit, threaded fit, or other methods. Through this method, the capsule 812 forms an inner wall and inner wall with a seal 200 adhered to the open end of the inner wall and create an inner sealed compartment. The shell, 812 would form an outer wall with an open end and closed end and upper compartment above the sealed compartment.

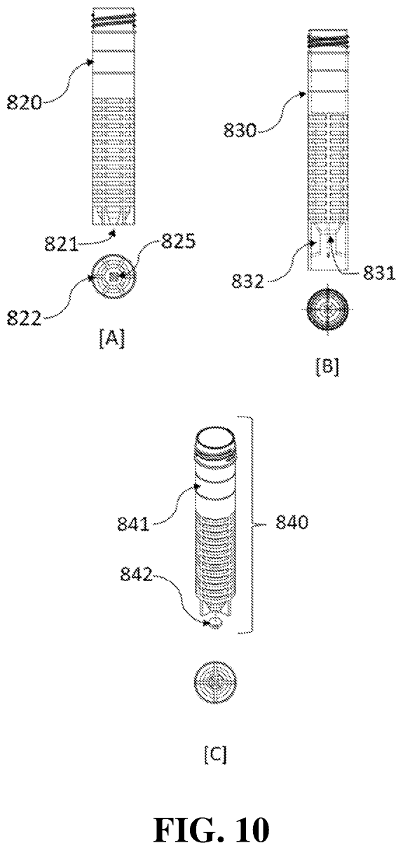

In FIG. 10. panels [A], [B], and [C] depict alternate embodiments of the SafeCollect® tube with additional features to accommodate certain methods of automation. In one embodiment 820 the false bottom region of the tube may be removed in favor of a flat bottom 821. In some embodiments, features may be added to the bottom of the tube to facilitate anti-rotation functionality in some automated decapping system 822. A variety of decapping systems exist, so anti-rotation features are subject to change based on application, platform, etc. In some cases, the bottom of the tube may include an identifying code 825 to identify the individual tubes (common codes include DataMatrix and QR codes). These codes could be read from the bottom using commercially available plate readers. As described for previous embodiments, the dimensions of the tube may be adjusted to suit market needs. For instance, in some embodiments, the false bottom design may be conserved 830. In such cases, the flat bottom of the tube may be on the interior of the false bottom 831. In some embodiments, antirotation features 832 can be located in the false bottom cavity. In other embodiments, slits could be added to the false bottom skirt to serve the antirotation function.

In some embodiments, the tube may be manufactured with an overmolded region on the bottom to add contrast to the code on the bottom 840. Overmolding is a process whereby two different plastics are used to produce a single final part. In one embodiment, the main body of the tube 841 may be composed of a transparent polypropyelene while the overmolded disk 842 would be a white HDPE. When the 2D code is applied, the readability of the code would be enhanced against a white background. Overmolding is a common method of plastic production, other embodiments could utilize overmolding in ways not described herein. For instance, the exterior surface of the SafeCollect® tube could be overmolded for decorative purposes or to fill in gaps left by the rib features of the tube.

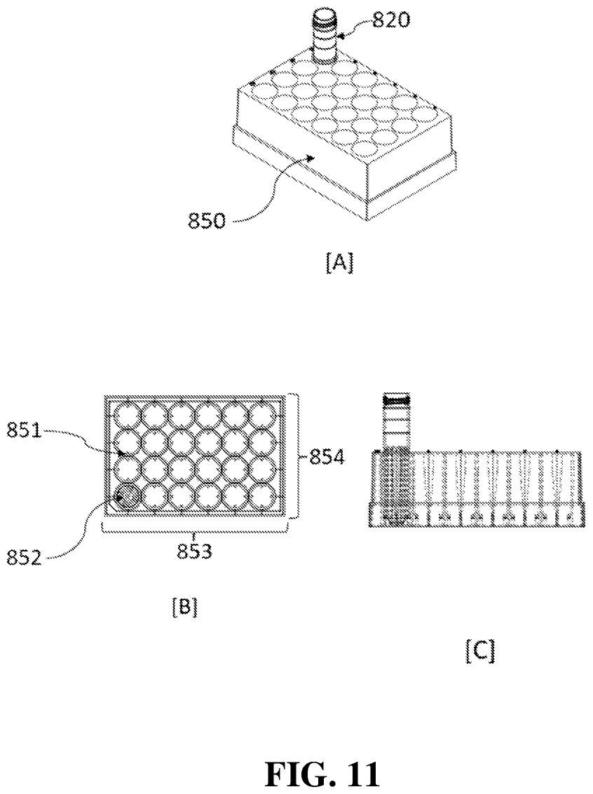

In FIG. 11 panels [A], [B], and [C] illustrate a way in which a tube 820 may interface with a rack 850 for storage, transport, and handling. In this embodiment, the rack dimensions will follows SBS format guidelines. The SBS format is a standardized plate dimension used in the biotech industry to standardize equipment and processes. The standardized plate dimensions define the length 853 and width 854 of the plate design. An SBS formatted plate could hold 24 tubes total in a 6×4 orientation. In some cases the rack may have a corresponding lid that covers the rack 850. In some embodiments, the rack lid may be attached to the rack via a hinge while in other embodiments the lid may be a separate part. Alternate rack designs and footprint dimensions may be developed to suit new equipment, or particular customer needs. In most cases, the rack 850 would include antirotation features 851 that would interface with the tube during decapping to inhibit continual rotation of the tube. The racks would also be open at the bottom 852 to allow for reading of a 2D code on the bottom of the tubes, as described in FIG. 10. In a racked format, some embodiments of the tube could be decapped on commercially available decappers designed to decap tubes in SBS format racks. Alternate rack designs may include antirotation features that extend into the bottom of a false bottom tube, such as the tube depicted in FIG. 10B. The use of antirotation features that interact on the interior surface of a false bottom tube have not traditionally been observed.

FIG. 12 Illustrates the piercing cap 300 used with the tube for liquid samples. The cap 300 is screwed onto the tube via threading 330 that corresponds to the threading 115 on the tube 100. In some embodiments of the cap 300, the threading profile 331 must allow for pop-off ejection during molding. In pop-off ejection, the thread profile 331 has a downward angle such that the threads 330 to not inhibit the mold from being pulled out of the part. Other embodiments may not necessitate pop-off ejection and could use thread profiles conducive to unscrew molding.

As the cap 300 is screwed onto the tube 100, a Sure Punch piercing element 310 that extends from the center of the cap 300 is lowered into the tube which pierces the seal 200. As the cap 300 is screwed onto the tube 100, a molded ledge 320 forms a press fit with the inner walls of the tube which prevents liquid from leaking out of the tube. This press fit is formed before the cap 300 is fully screwed onto the tube 100, inhibiting leakage from the tube even if the user fails to fully screw the cap onto the tube. The ledge 320 is but one method of producing a press fit between the tube/cap interface, and alternate designs could be utilized to achieve a leakproof seal between the tube 100 and the cap 300.

The Sure Punch piercing element has a number of features to pierce the seal 200 and allow the liquid or material in the upper compartment 160 of the tube 170 to mix with the stabilizing reagent in contained in the lower sealed compartment 170 of the tube. End 312 of the Sure Punch piercing element 310 has an angled surface 313 to form an edge. As the Sure Punch piercing element is lowered into the tube 100, the end 312 applies pressure to the seal 200 to form an initial cut in the seal, this occurs before the cap 300 is fully screwed onto the tube. Applying further rotation to the cap 300 causes end 312 to continue to cut along the foil seal 200 to form a circular punch that is pushed to the side of the tube by the Sure Punch piercing element design. The length of the Sure Punch piercing element 311 prevents the piercing process from occurring until the cap 300 is being screwed onto the tube and the press fit has been formed by the molded ledge 320. In some preferred embodiments, the preferred lengths 311 of the Sure Punch piercing element is in the range of about 25 mm to about 26 mm. Other embodiments may lengthen the piercing element to allow for the seal 200 to be pierced as the cap 300 is placed onto the tube 100, prior to engagement of the threads 330. Other embodiments may adjust the length 311 of the Sure Punch piercing element to suit a specific functional or decorative purpose. The Sure Punch piercing element is cylindrical and hollow 315 to minimize the overall volume of the piercing element, and to allow for efficient mixing. The Sure Punch piercing element also has slots cut into the side 314 to further facilitate mixing of the reagents in the tube 100. The incorporation of angled end 313 in piercing element 310 provides a mechanism to

The top of the cap 300 has a diameter 350 and height 340 optimized towards automated decapping of the cap. The cap 300 also includes an array of grips 341 to aid in screwing on the cap manually. Other embodiments of the piercing cap 300 may alter the cap diameter 350 or height 340.

FIG. 13. Shows the result of a piercing cap 300 that has been fully screwed onto a sealed SafeCollect® tube 100. The engagement of the cap 300 results in a pierced seal 201 which formed a foil punch at the side of the tube 100. This allows the liquids in the upper 160 and lower 170 compartments of the tube 100 to mix efficiently.

FIG. 14 panels [A] and [B] depict alternative embodiments of the piercing cap design that incorporate additional functionality. In FIG. 14A, one embodiment of the cap 303 has a modified piercing element 360 that can act as a scoop that can add various samples to the collection device. Modified piercing element 360 maintains the piercing functionality of the Sure Punch piercing element described previously 310 but can also be used to transfer soil, feces, or other sample types to the device. The overall length over the modified piercing element 360 is the same as the previously described length 311 for the Sure Punch piercing element. Instead of the fully circular design of the Sure Punch piercing element 310, modified piercing element has a semi circle design while performing the same piercing functionality. In an alternative embodiment of the cap 302 the tip of the piercing element 361 is now tapered to form a point while still performing the piercing functionality described previously in the Sure Punch piercing element 310. FIG. 14A depicts but two exemplary alternative designs for the Sure Punch piercing element 310, a number of alternative piercing element can be developed for functional or decorative purposes while performing the seal piercing function of the cap 300 depicted in FIG. 12.

FIG. 14B depicts an alternative cap 304 with features 380 added to the top of the cap to allow for automated decapping with bits that insert into the cap (as opposed to grippers). Due to the variety of options available for automated decapping, these features can include (but are not limited to) ribs, adjusting the top profile of the cap, and others. Similar features could also be incorporated into the swab cap, 500, or other cap designs to be used with SafeCollect® tube 100.

In FIG. 15 panels [A] and [B] depict additional embodiments of the piercing cap 300 designed for more precise sample collection of specific sample types. FIG. 15B shows an alternate embodiment of the piercing cap 306. In this embodiment the piercing element 375 has been modified to form a scoop at the tip 376 such that the cap could be used to scoop up a defined volume of sample before screwing it into the cap. A number of different embodiments of the cap could be used suited towards different sample types (e.g., fecal samples, soil samples, etc.). A side view of the cap is shown in the view on the left, while a section view taken along the centerline of the cap is shown in the view on the right.

In addition to a scooping function, the cap can also be modified to collect samples via alternative methods. For instance, FIG. 15A depicts two views of a cap 305 that collects a defined sample volume by plunging the modified piercing element 370 into a sample and rotating the cap to core the sample. The piercing element has been modified to by adding an enclosed cavity 371 with an open structure, sized to hold of a specific volume, to the end of the piercing element. In the cap embodiment 305, the piercing element 370 has been modified to collect one gram of fecal material in the interior cavity of the cap 371. Such designs can be adjusted to collect different amounts of material, depending on the application. For example, instead of one gram, the design of the piercing element could be adjusted to collect 100 mg, 200 mg, 300, 400, or 500 mg of fecal material instead of one gram. Alternate embodiments could also collect over 1 g of material, including up to 10 g of material. The coring principle for sample collection depicted in FIG. 15A could be expanded to sample types other than feces, such as clay, soil, and more.

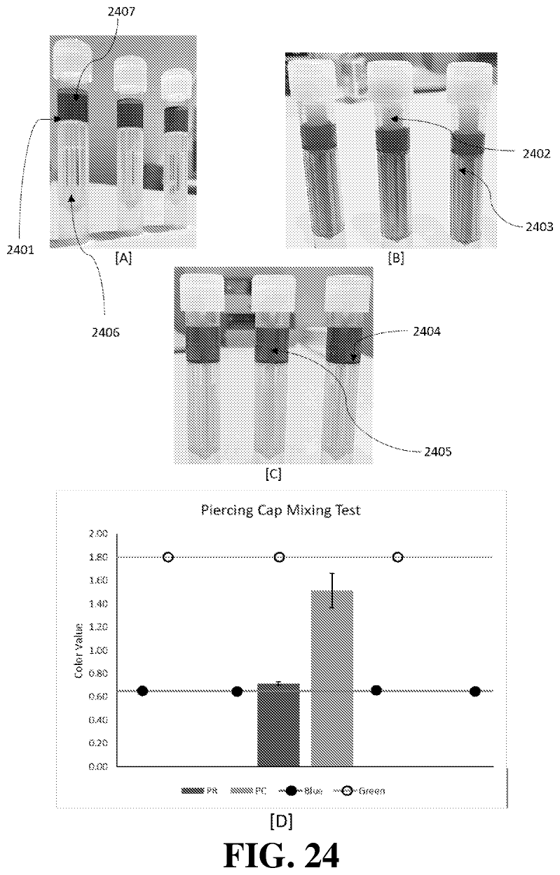

FIG. 24 presents a specific example of the piercing cap 300 with the unique Sure Punch piercing element design functionality in comparison to alternative designs that could be considered that does not utilize the Sure Punch piercing element. In this experiment, FIG. 24A shows SafeCollect® tubes that were prepared with two differently colored media. The lower chamber of the device contains a yellow media 2406, representing a stabilization reagent, sequestered by an intact seal, 2401. Atop the seal is a blue media 2407 that was added the SafeCollect® device, representing saliva or some other liquid sample. Multiple tubes were prepared and the tubes were then capped with one of two caps, and then inverted once to mix the blue 2407 and yellow 2406 media. One cap was the piercing cap (PC) depicted in FIG. 12, utilizing the unique Sure Punch piercing element design, developed for the specific application with SafeCollect® tubes to facilitate efficient mixing between the upper compartment 160 and lower sealed compartment 170. The alternative cap was a swab cap 500 into which a narrow rod (of about 2.5 mm in diameter) was inserted into the cap where the swab end 620 would usually be inserted, extending from the center of the cap into the SafeCollect® device. The cap that incorporated the narrow rod to perform the piercing is referred to as the piercing rod (PR).

The FIG. 24B depicts devices capped with the piercing cap 300. In the figure the Sure Punch piercing element 2402 has pierced the seal 200, resulting in a punchout 2403 which is pressed to the side of the tube. FIG. 24C displays devices capped with caps that had the piercing rod feature, 2405. The piercing rod feature does pierce the seal 2404 but much of the seal remains intact and the piercing rod blocks the flow of media between the upper and lower compartment of the device.

FIG. 24D further describes the efficacy of the piercing cap (PC) 300 compared to the piercing rod (PR) by assigning a color value to media drawn from the top compartment 160 of each device after capping and inversion. As shown, the piercing cap 300 results in a significantly more thoroughly mixed media close to the green color value (a very thoroughly mixed sample of the blue and yellow media that were used in the experiment). In contrast, the prior art piercing rod inhibits mixing of the two media, despite piercing the seal, and the color value for the top compartment of the device is blue indicating that no mixing between the upper compartment 160 and lower sealed compartment 170 has occurred. The unique and efficient design of the piercing cap 300 and Sure Punch piercing element 310 resulted in a color value shift of about 75% between the unmixed blue and the perfectly mixed green. Conversely, the piercing rod design only result in a 5% color shift towards the perfectly mixed green. Based on this data, the Sure Punch piercing element conferred an improvement in mixing efficiency of over 15× what could be accomplished using the piercing rod design.

The data presented in FIG. 24 highlights the importance and uniqueness of the Sure Punch piercing element design features described for the piercing cap 300 (FIG. 12). While a variety of designs may exist that could pierce the seal in the SafeCollect® device (e.g. the piercing rod cap), only a subset of these designs will allow for sufficient mixing of the two compartments. Sufficient mixing is an important feature of the device for liquid samples; liquid samples that are not adequately mixed with the stabilization reagent will not be preserved as reliably as well mixed samples.

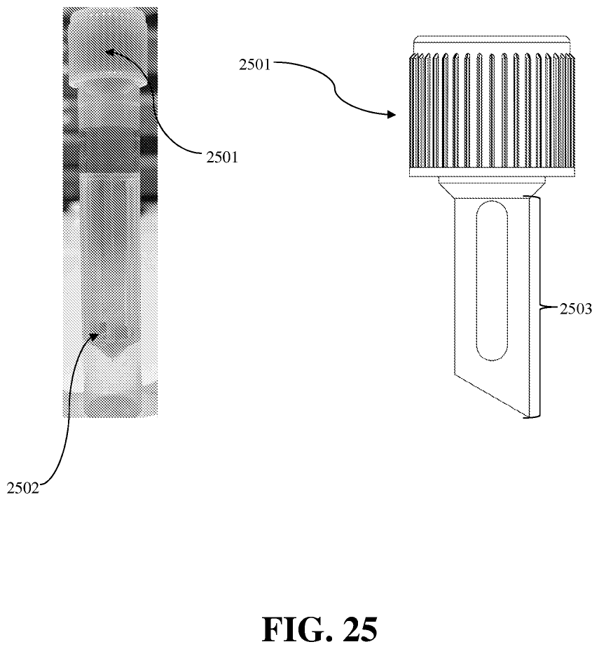

FIG. 25 depicts an experiment with a modified version of the piercing cap 2501 with an extended piercing element 2503, wherein the Sure Punch piercing element 310 length 311 has been increased beyond the preferred 25-26 mm. This version of the cap 2501 results in the foil of the seal being completely punch out, resulting in a freefloating disc 2502 in the device. The freefloating disc is undesirable and detrimental to the function of the device. The disc can inhibit mixing by blocking the flow between the two chambers of the device. Furthermore, the disc can interfere with downstream processes for the device, such as pipetting liquid out of the device. Despite a general design similarity to the piercing cap 300 and Sure Punch piercing element 310 described in FIG. 12, this experiment shows that the extended piercing element 2503 has a detrimental impact on cap function. This further highlights the uniqueness and superior functionality of the piercing cap design, and the features of the Sure Punch piercing element, and its interaction with the internally sealed SafeCollect® device. While a variety of designs could pierce the seal, only a subset of designs can pierce the seal in a manner that allows for sufficient mixing between the upper and lower chambers of the SafeCollect® device.

As shown in FIG. 17, in some embodiments, the SafeCollect®™ tube is used for the collection of swab samples as opposed to saliva or other liquid samples. In such embodiments, a different type of cap is used than the piercing cap described for other embodiments. The swab cap 500 is for use with swab samples and retains the swab 600 during the decapping process.

As with embodiments of the piercing cap 300, the swab cap has a diameter 530, height 540, and grips 541 optimized towards automated decapping. Alternate embodiments may adjust the diameter 530 and height 540 of the cap 500. The threading 550 of the swab cap 500 corresponds to the threading 115 of the tube 100. In some embodiments, the threading profile may be designed towards an unscrew mold ejection method. Alternate embodiments may use thread profiles that allow for pop-off ejection during the injection molding process. The center 510 of the swab cap 500 inserts into the tube 100. As the cap 500 is placed onto a tube 100 containing a swab 600, an angled feature 511 pushes the swab end 620 towards the center channel of the cap 512. As the swab end 620 is pushed into the center 510 of the cap 500, and array of fins 520 produce a press fit between the swab 600 and the cap 500. The fins 520 are thin with a pointed tip 521 to accommodate common swab diameters. The fins 520 are angled at the bottom 522 to aid in centering the swab end 620. Other embodiments of the cap 500 may adjust the design or layout of the retention fins 520. When a swab 600 has been centered into the swab cap 500, the press fit formed between the end of the swab and the cap allow the cap to be removed from the tube 100 while retaining the swab. This is important for use of the tube 100, a swab 600 that is left in the tube could inhibit access to the stabilization reagent at the bottom of the tube 100, which can negatively impact downstream processing. By retaining the swab 600 in the cap 500, the swab is removed in the same step as the tube 100 is decapped. In alternate workflows a swab 600 in the tube 500 may have no negative impact on processing of the tube in a laboratory, and a standard cap that does not capture the swab may be favored over a swab capture cap 500 design.

FIG. 18 contains a general representation of a swab 600 that can be used to collect a sample with the SafeCollect®™ device. The swab has a collection head 610 which can be composed of various materials depending on the embodiment (nylon, PE, etc.). In some embodiments, the swab head may be a flocked swab design while others may not be flocked. Here, flocked refers to a method of swab production wherein microfibers of polymer materials are coated onto the swab head 610 orthogonal to the surface of the swab head. Flocked swabs are used widely in the industry as a robust, consistent method of collecting samples with a swab based collector. One alternative to flocked swabs is a woven swab wherein polymers are wrapped around the swab head to form the collector. The dimensions of the swab head 610 may vary based on application or supplier. The swab head 610 is attached to the swab shaft 630. In many embodiments, the swab shaft 630 is approximately 80 mm in length. In one embodiment, the bottom of the tube 190 is equivalent to the length of the swab 600 to facilitate the use of the swab cap 500. A short swab that doesn't not reach the top of the tube will not be retained by the swab cap 500. The end of the swab 620 fits into the center of the swab cap 500. For the use of a swab cap 500, the diameter of the swab should be of a sufficient diameter to fit tightly into the central channel 512 of the swab cap 500. In many embodiments, the end of the swab 620 is formed by a breakpoint swab, which has a notched breakpoint that allows for longer swab designs that can be broken to produce the desired swab length for interfacing with the swab cap. In many embodiments, the SafeCollect®™ tube can accommodate breakpoint swabs from various suppliers. Many swabs used in the industry have similar lengths, diameters, and designs. The description herein is only an example of swabs that could be used with the device.

FIG. 19 depicts alternate designs that may be used with the SafeCollect®™ tubes and swab caps. One concept to be included are shafts tipped with an adhesive collector. These adhesive collectors could be used to collect samples from the skin or other surface with high levels of retention as compared to non-adhesive swabs. For instance, some embodiments such as 601 and 602 have a mechanical roller coated in an adhesive such that the tip is rolled over the surface of interest to collect a sample. Still other embodiments, such as 603 have a cylindrical head coated in adhesive which can be rolled over the surface manually. Yet other embodiments, such as 604 are flat and can be touched to the surface of interest to collect a sample.

The inclusion of an adhesive collection apparatus with a collection device is not yet observed. This is a novel method for collection samples from skin or other surfaces where traditional swabs are not sufficient to pick up all material on the given surface. The adhesive collector designs are not constrained to the embodiments described herein.

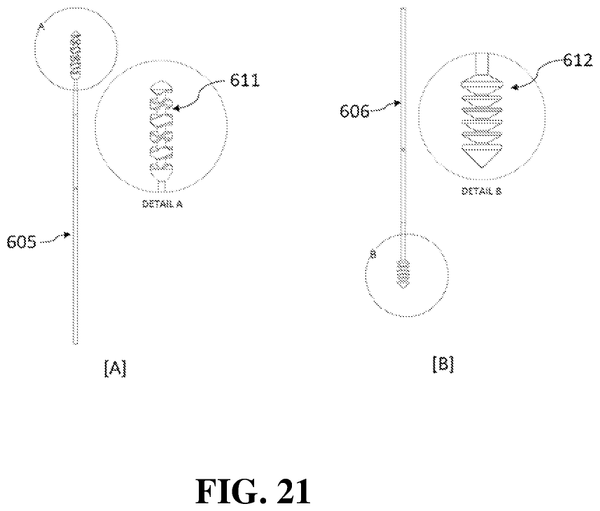

Swab-type designs for collecting a defined volume of sample (either liquid or solid) would also be usable with the SafeCollect® sample collection device design. FIG. 21, panels [A] and [B] represent various designs for the collection of defined sample sizes. In one embodiment, a sample collector 605 has a grooved collection head, 611, for collecting mucosal samples from the nostril. In another embodiment, a sample collector 606 has a collection head with an angular step design, 612, for collecting a pre-defined mass of fecal samples. The designs depicted in FIG. 21 are just one example of defined volume sample collection using a swab-type collector. The designs depicted in FIG. 21 function by capturing material in the molded grooves of the collector head. These types of collectors are viable with a variety of different sample types, such as solid fecal material, viscous mucous or similar liquids, but also non viscous liquids such as water. The dimensions and profile of the collectors heads 611 and 612 can be altered to collect larger (or smaller) volumes or be tailored to specific sample types (e.g. solid vs loose fecal samples). Another benefit of these designs is that they can be manufactured via injection molding or 3D printing, without the need for the complex methods required to produce flocked or woven swabs. A variety of collectors could be developed for specific samples, with different designs and profiles at the swab head that are highly specialized. Any such design could be incorporated into a SafeCollect® sample collection device where in the sample is submerged into a stabilization reagent sequestered by a pierceable seal.

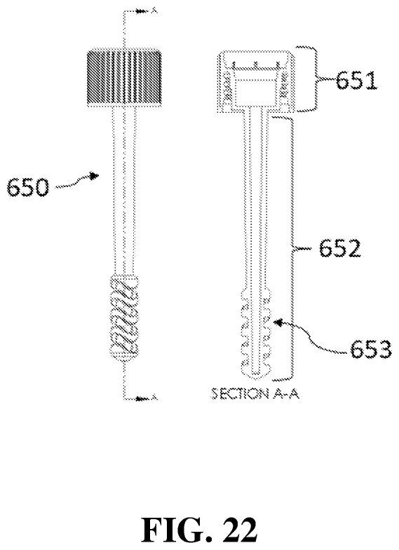

FIG. 22 depicts an alternate cap and swab embodiment 650 wherein the cap and swab are combined into a single part. In this embodiment, the part is composed of two components, an upper cap portion 651, and a lower swab portion, 652. The cap portion, 651, serves some of the same cap functions as other cap embodiments described (i.e. threaded closure to contain the reagent and sample in the device during transport). The swab portion is directly attached to the to the cap portion and serves the same sample collection functionality as other swab collectors described in this document. For the cap embodiment presented in FIG. 22, the head of the swab 653 is just one example of the collector design that could be incorporated into the combine cap/swab design. Alternative collectors could include, but are not limited to, flocked swabs or other collectors depicted in FIGS. 19 and 21.

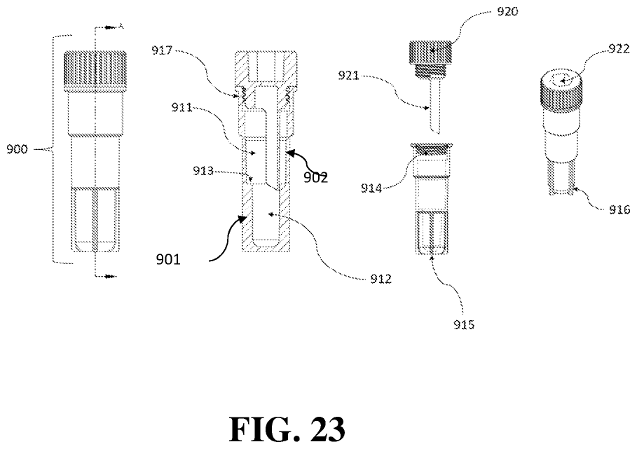

As described previously, the SafeCollect® internal sequestration design concept can be expanded to suit a variety of different sample types by adjusting the dimensions of the device, specific to new applications. For instance, in FIG. 23, the SafeCollect® design has been miniaturized to collect small amounts of blood (as opposed to saliva, swabs, etc. described previously.) In this embodiment, the device 900 is composed of a sequestered tube 910 and piercing cap 920. Tube 910 comprises a cylindrical tube with an open end and closed end. The open end of tube 910 is proximal to an outer wall portion 902 and the closed end is proximal to an inner wall portion 901. The open end of inner wall portion 901 is coupled to the outer wall portion 902 forming internal surface 913 as the open end of the inner wall portion. As seen in prior embodiments described, the tube design utilizes an interior compartment 912 sealed by a pierceable membrane, bonded to the internal surface 913. The sequestered compartment 912 would contain a reagent to stabilize a small volume of blood collected. In some embodiments the upper section of this tube 917 interfaces with a capillary blood collection device to collect a small amount of blood in the upper chamber of the tube 911. The tube would be removed from the blood collecting device to be capped. Similar to some other embodiments of the SafeCollect® design, a piercing cap 920 with a piercing element 921 is then screwed onto the tube to pierce the seal allowing the blood to mix with the reagent contained in the sequestered cavity. In specific embodiments, the piercing element has a length of 15-16 mm to pierce the seal in tube 910. In this embodiment, internal threading in the tube 914 is used to interface with the cap as opposed to external threading found in other embodiments described previously. It should be noted that this embodiment includes other features discussed previously, such as a flat bottom for 2D coding 915 and anti-rotation features on the tube 916 and the cap 922. This specific application represents but one scenario wherein the SafeCollect® design can be adapted (e.g. adjusting the dimensions, threading, device profile, etc.) to fit a novel application.

The embodiment illustrated in FIG. 23 was developed to interface with a blood collection device at the opening of the tube 917. In certain embodiments, the tube 910 is provided separately to interface with a blood collection apparatus. In other embodiments, the tube 910 may be provided already attached to a blood collection apparatus. The illustrated embodiment was design for a capillary blood collection device wherein small blades puncture the skin and draw blood from the surface capillaries of the skin into the tube 910. A number of suppliers exist for capillary blood collection devices exist and the opening 917 of the tube 910 could be adjusted to interface with specific blood collection devices. In additional to capillary blood collection devices, the SafeCollect® could be adjusted to fit alternative to interface with alternative blood collection methods including, but not limited to, lancet collection or collection via a syringe or venous puncture.

Adaptation of the SafeCollect® design to novel applications (e.g. blood) may necessitate adjustments to the preferred dimensions described in the illustrated embodiments. This may entail alterations to the diameter D and height 109 of the illustrated tube 100, and adjustments to or removal of the rib pattern 180. Corresponding adjustments may be made to the Sure Punch piercing element 310 design, including shortening or lengthening of the piercing element lengths. The designs and methods disclosed in this document represent a subset of applications for the invention. Designs or methods that deviate from the preferred designs or methods disclosed may be derived from the SafeCollect® device design. Particularly, the invention of an internally sealed sample collection tube, containing a stabilization reagent, and a coupling of that tube with a piercing cap (and corresponding piercing element) or be pierced by a swab-type collection device. The tube could be manufactured via injection molding as a single part with an internal sealing surface for depositing a seal. Alternatively, a sealed capsule could be placed into an outer shell to produce a collection tube with an internally sealed compartment.

Referring to FIG. 4C, the sample or specimen 450 is preferably stabilized for transport in a specimen transport medium, viral transport medium, or other medium. Specimen or viral transport mediums (VTM) should stabilize and preserve DNA/RNA for downstream biological analysis. Transport mediums are generally one of three main categories: lysis reagents, direct amplification reagents, and general transport media (e.g., saline buffers). A preferred lysis reagent, DNA/RNA Shield™, inactivates pathogens (e.g., fungi, bacterial, viral) and allows isolating nucleic acid directly without precipitation or reagent removal. The ability to inactivate pathogens is a preferred safety measure for both the consumer and lab workers. Direct amplification reagents allow analysis by polymerase chain reaction (PCR) and other means by amplification without extraction or purification. Direct amplification creates a more streamlined and efficient workflow eliminating the need for extraction or purification. Direct amplification works best with viral specimens, but analysis can become difficult with tissue or complex samples. Transport mediums generally comprise of buffered saline solutions, but do not provide for added stabilization of the sample's DNA/RNA. Examples of general transport mediums include, but are not limited to, viral transport mediums (VTM), universal transport mediums (UTM), general transport medium (GMT), and clinical virus transport mediums (CTM).

Lysis based specimen and VTM reagents allow for the chemical stabilization of nucleic acids due to chaotropic or detergent-based components, or other components. Additionally, lysis reagents can be modified to consist of chaotropic agents, chelating agents, surfactants, ribonuclear inhibitors, visual indicators, mucolytic agents, alcohol, antimicrobials, buffers, and acids. Clinical specimens suitable for use with specimens and VTMs include, but are not limited to, saliva, blood, sputum cultures, stool, urine, nasopharyngeal discharges, mucosa! surfaces, vesicular lesions, conjunctival/ocular, throat, rectal, cervical, bodily fluids, cerebrospinal fluid (CSF) samples.

Commercially available lysis reagent viral transport mediums include, but are not limited to, DNA/RNA Shield™ (Zymo Research Corporation), PrimeStore®Molecular Transport Medium (Longhorn Vaccines & Diagnostics LLC), SDNA-1000 (Spectrum Solutions), DNAgard® (Biomatrica, Inc.), Oragene®Dx, OMNigene® (DNA Genotek Inc., and SideStep Lysis and Stabilization Buffer (Agilent Technologies, Inc.). Commercially available direct amplification viral transport mediums include, but are not limited to, DNA/RNA Shield DirectDetect™ (Zymo Research Corporation), XpressAmp™ Direct Amplification Reagents (Promega Corporation), DirectDetectTMSARS-COV-2 Direct Real-time RT-PCR for Environmental Studies (PerkinElmer Inc.), and FastAmp® (Intact Genomics, Inc.). Commercially available general transport media include, but are not limited to UTM® Universal Transport Medium (Copan Diagnostics, Inc.) and Puritan UTM-RT Collection and Transport System (Puritan Medical Products LLC).

All of the devices and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are incorporated herein by reference:
U.S. Pat. No. 4,843,155
U.S. Pat. No. 4,985,026
U.S. Pat. No. 6,821,789
U.S. Pat. No. 6,458,546
U.S. Pat. No. 6,602,718
U.S. Pat. No. 6,617,170
U.S. Pat. No. 6,913,932
U.S. Pat. No. 7,682,790
U.S. Pat. No. 8,084,443
U.S. Pat. No. 8,293,467
U.S. Pat. No. 8,669,240
U.S. Pat. No. 8,415,330
U.S. Pat. No. 9,732,376
U.S. Pat. No. 10,525,473
U.S. Pat. No. 10,774,368
U.S. Pat. No. 10,799,422
U.S. RE43389
U.S. Pat. Pub. 20120052572 EP0366826A1
PCT Pat. Pub. WO1995035390

What is claimed is:

1. A device for collecting a sample, the device comprising:
a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
a plurality of ribs coupling the first outer wall portion and the second outer wall portion to the inner wall portion, wherein:
the plurality of ribs comprises a plurality of central ribs each having planar sides and a curved end;
the plurality of ribs comprises a first outer rib having a flat side and a curved side; the plurality of ribs comprises a second outer rib having a flat side and a curved side;
the plurality of ribs comprises a first perpendicular rib coupled to the flat side of the first outer rib and the inner wall portion; and
the plurality of ribs comprises a second perpendicular rib coupled to the flat side of the second outer rib and the inner wall portion;
the inner wall portion comprises a closed end proximal to the second end of the cylindrical tube; and
the inner wall portion comprises an open end proximal to the first end of the cylindrical tube; and
a pierceable seal coupled to the open end of the inner wall portion.

2. The device of claim 1 further comprising a cap configured to couple to the first end of the cylindrical tube.

3. The device of 2 further comprising a sample collector coupled to the cap.

4. The device of claim 3 wherein the sample collector as a grooved collection head.

5. The device of claim 3 wherein the sample collector as a collection head with an angular step design.

6. The device of claim 3 wherein the sample collector as a flocked, woven, or sponge swab.

7. The device of claim 3 wherein the sample collector as an adhesive collection head.

8. The device of any one of claims 1-7 wherein the inner wall portion, pierceable seal and tapered closed end form a sealed compartment.

9. The device of claim 8 wherein the sealed compartment comprises a reagent.

10. The device of any one of claims 1-9 wherein each central rib is tapered such that a thickness of each central rib at the curved end is less than a thickness of each central rib proximal to the inner wall portion.

11. The device of any one of claims 1-10 wherein each planar side of each central rib is tapered 1 degree.

12. The device of any one of claims 2-11 wherein the cap is configured to couple to the first end of the cylindrical tube via a threaded coupling.

13. The device of any one of claims 2-12 wherein the cap comprises a piercing element configured to pierce the pierceable seal when the cap is coupled to the first end of the cylindrical tube.

14. The device of claim 13 wherein the piercing element has a circular or semi-circular cross-section.

15. The device of claim 14 wherein the circular or semi-circular cross-section is hollow.

16. The device of claim 15 wherein the piercing element has an end with an angled surface.

17. The device of any one of claims 13-16 wherein the cap comprises a ledge configured to seal to the cylindrical tube.

18. The device of any one of claims 13-17 wherein the piercing element has a length of approximately 25 mm to approximately 26 mm.

19. The device of any one of claims 13-18 wherein the cap comprises threads configured to engage the cylindrical tube.

20. The device of any one of claims 17-19 wherein the ledge is configured seal to the cylindrical tube prior to the piercing element piercing the pierceable seal when the cap is threadably engaged with the cylindrical tube.

21. The device of claim 14 wherein the circular or semi-circular cross-section is configured to form a circular cutout of the pierceable seal.

22. The device of claim 21 wherein an arc of about 20 to about 45 degrees is attached to the sealing surface.

23. The device of claim 21 wherein the piercing element comprises an angled surface configured to push aside the circular cutout.

24. The device of any one of claims 1-23 wherein the plurality of ribs comprise lateral ribs.

25. The device of claim 24 wherein the plurality of ribs form a checkerboard pattern.

26. The device of any one of claims 1-25 wherein the ribs are eliminated in favor of a gap between the inner wall portion and outer wall portion to form the sealing surface.

27. The device of any one of claims 1-26 wherein the closed end is tapered.

28. The device of any one of claims 1-27 wherein the closed end comprises a flat bottom.

29. The device of any one of claims 1-28 wherein the closed end comprises an identifying code.

30. The device of any one of claims 1-29 further comprising a tube capsule inserted into the cylindrical tube.

31. The device of claim 30 wherein the tube capsule is filled with a reagent.

32. The device of claim 30 or 31 wherein:
the tube capsule comprises an inner wall portion and an outer wall portion; and
the outer wall portion of the tube capsule is inserted into the inner wall portion of the cylindrical tube.

33. The device of any one of claims 30-32 wherein the tube capsule comprises an open end and closed end.

34. The device of claim 34 wherein the open end of the tube capsule is sealed

35. The device of claim 35 wherein the open end of the tube capsule is sealed via ultrasonic welding, laser welding, adhesives, press fit, or threaded fit.

36. A method of collecting a sample, the method comprising:
obtaining a device for collecting the sample, the device comprising:
a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
a plurality of ribs coupling the first outer wall portion and the second outer wall portion to the inner wall portion, wherein:
the plurality of ribs comprises a plurality of central ribs each having planar sides and a curved end;
the plurality of ribs comprises a first outer rib having a flat side and a curved side; the plurality of ribs comprises a second outer rib having a flat side and a curved side;
the plurality of ribs comprises a first perpendicular rib coupled to the flat side of the first outer rib and the inner wall portion; and
the plurality of ribs comprises a second perpendicular rib coupled to the flat side of the second outer rib and the inner wall portion;
the inner wall portion comprises a tapered closed end proximal to the second end of the cylindrical tube; and
the inner wall portion comprises an open end proximal to the first end of the cylindrical tube;
a pierceable seal coupled to the open end of the inner wall portion; and
a cap configured to couple to the first end of the cylindrical tube; and
depositing a sample into the first end of the cylindrical tube; and coupling the cap to the first end of the cylindrical tube.

37. The method of claim 36 wherein:
the device further comprises a funnel; and
depositing the sample into the first end of the cylindrical tube comprises:
coupling the funnel to the first end of the cylindrical tube; and
depositing the sample into the funnel.

38. The method of claim 36 or claim 37 wherein:
the cap comprises a piercing element configured to pierce the pierceable seal when the cap is coupled to the first end of the cylindrical tube; and
coupling the cap to the first end of the cylindrical tube pierces the pierceable seal.

39. The method of claim 38 wherein:
the inner wall portion contains a reagent between the pierceable seal and the tapered closed end; and
coupling the cap to the first end of the cylindrical tube pierces the pierceable seal and mixes the sample with the reagent.

40. The method of claim 39 further comprising inverting the cylindrical tube to further mix the sample with the reagent.

41. The method of claim 39 or claim 40 wherein the reagent stabilizes the sample.

42. A method of manufacturing a cylindrical tube, the method comprising:
injecting a liquid polymer at a first temperature into a mold partially comprised of a split mold, wherein the split mold comprises a first half and a second half coupled at a split line;
reducing the first temperature of the liquid polymer injected into the split mold to a second temperature, wherein the liquid polymer injected into the split mold forms a solid polymer component at the second temperature;
separating the first half of the split mold and the second half of the split mold at the split line;
removing the solid polymer component from the split mold, wherein the solid polymer component is a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
a plurality of ribs coupling the first outer wall portion and the second outer wall portion to the inner wall portion, wherein:
the plurality of ribs comprises a plurality of central ribs each having planar sides and a curved end;
the plurality of ribs comprises a first outer rib having a flat side and a curved side; the plurality of ribs comprises a second outer rib having a flat side and a curved side;
the plurality of ribs comprises a first perpendicular rib coupled to the flat side of the first outer rib and the inner wall portion; and
the plurality of ribs comprises a second perpendicular rib coupled to the flat side of the second outer rib and the inner wall portion;
the inner wall portion comprises a tapered closed end proximal to the second end of the cylindrical tube; and
the inner wall portion comprises an open end proximal to the first end of the cylindrical tube.

43. A device for collecting a blood sample, the device comprising:
a cylindrical tube comprising:
a first open end;
a second closed end;
an outer wall portion proximal to the first open end of the cylindrical tube;
an inner wall portion proximal to the second closed end and coupled to the outer wall portion, wherein the inner wall portion comprises an open end proximal to the first end of the cylindrical tube; and
a plurality of ribs extending from the closed end of the inner wall portion to the outer wall portion; and
a pierceable seal coupled to the open end of the inner wall portion.

44. The device of claim 43 further comprising a cap comprising a hollow circular or semi-circular piercing element configured to pierce the pierceable seal when the cap is coupled to the first end of the cylindrical tube, and wherein the piercing element has a length of approximately 15 mm to approximately 16 mm.

45. The device of claim 44 wherein the piercing element has an end with an angled surface.

46. The device of claim 45 wherein:
the piercing element is configured to form a cutout in the pierceable seal when the cap is coupled to the first end of the cylindrical tube; and
the angled surface is configured to push aside the cutout in the pierceable seal.

47. The device of claim 45 wherein the hollow circular or semi-circular piercing element is configured to form an approximately circular cutout of pierceable seal, wherein an arc of about 20 to about 45 degrees is attached to the sealing surface.

48. A device for collecting a fecal sample, the device comprising:
a cylindrical tube comprising:
a first open end;
a second closed end;
a first outer wall portion spanning the first and second end of the cylindrical tube;
a second inner wall comprising a cylindrical tube comprising:
a first open end;
a second closed end;
a wall spanning the first and second end; and
the cylindrical tube further comprising an inner wall portion inserted into the cylindrical tube comprising the outer wall portion, wherein the inner wall portion is coupled to the outer wall portion of the cylindrical tube,
a pierceable seal coupled to the open end of the cylindrical tube comprising the inner wall portion.

49. The device of claim 48 further comprising a cap configured to couple to the first end of the cylindrical tube, wherein the cap further comprises a sample collector coupled to the cap.

50. The device of claim 49, wherein the sample collector is a spoon or scoop.

51. A device for collecting a fecal sample, the device comprising: a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
a plurality of ribs coupling the first outer wall portion and the second outer wall portion to the inner wall portion, wherein:
the plurality of ribs comprises a plurality of central ribs each having planar sides and a curved end;
the plurality of ribs comprises a first outer rib having a flat side and a curved side;
the plurality of ribs comprises a second outer rib having a flat side and a curved side;
the plurality of ribs comprises a first perpendicular rib coupled to the flat side of the first outer rib and the inner wall portion;
the plurality of ribs comprises a second perpendicular rib coupled to the flat side of the second outer rib and the inner wall portion;
the inner wall portion comprises a closed end proximal to the second end of the cylindrical tube;
the inner wall portion comprises an open end proximal to the first end of the cylindrical tube;
a pierceable seal coupled to the open end of the inner wall portion;
a wall spanning the first and the second end; and
the cylindrical tube further comprising an inner wall portion inserted into the cylindrical tube comprising the outer wall portion, wherein the inner wall portion is coupled to the outer wall portion of the cylindrical tube,
a pierceable seal coupled to the open end of the cylindrical tube comprising the inner wall portion.

52. The device of claim 51 further comprising a cap configured to couple to the first end of the cylindrical tube, wherein the cap further comprises a sample collector coupled to the cap.

53. The device of claim 52, wherein the sample collector is a spoon or scoop.

54. A device for collecting a sample, the device comprising:
a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
the inner wall portion comprises a closed end proximal to the second end of the cylindrical tube; and
the inner wall portion comprises an open end proximal to the first end of the cylindrical tube; and
a pierceable seal coupled to the open end of the inner wall portion and a gap between the inner wall portion and outer wall portion to form the sealing surface.

55. A device for collecting a sample, the device comprising:
a cylindrical tube comprising:
a first end;
a second end;
a first outer wall portion proximal to the first end of the cylindrical tube;
a second outer wall portion proximal to the second end of the cylindrical tube;
an inner wall portion extending between the first outer wall portion and the second outer wall portion; and
the inner wall portion comprises a closed end proximal to the second end of the cylindrical tube; and
the inner wall portion comprises an open end proximal to the first end of the cylindrical tube; and
a pierceable seal coupled to the open end of the inner wall portion a gap between the inner wall portion and outer wall portion to form the sealing surface; and
a tube capsule inserted into the cylindrical tube.

56. The device of claim 55 wherein the tube capsule is filled with a reagent.

57. The device of claim 55 or 56 wherein:
the tube capsule comprises an inner wall portion and an outer wall portion; and
the outer wall portion of the tube capsule is inserted into the inner wall portion of the cylindrical tube.

58. The device of any one of claims 55-57 wherein the tube capsule comprises an open end and closed end.

59. The device of claim 58 wherein the open end of the tube capsule is sealed

60. The device of claim 58 wherein the open end of the tube capsule is sealed via ultrasonic welding, laser welding, adhesives, press fit, or threaded fit.
